SEQUENCE LISTING (42 sequences)

SEQ ID NO: 1 - DNA, 22 bp, Artificial Sequence
  Description of Artificial Sequence Synthetic primer
tcacagtcactgacaccaacga

SEQ ID NO: 2 - DNA, 20 bp, Artificial Sequence
  Description of Artificial Sequence Synthetic primer
ggcacctgacccttgtacgt

SEQ ID NO: 3 - DNA, 19 bp, Artificial Sequence
  Description of Artificial Sequence Synthetic primer
tgccgctttgcaggtgtat

SEQ ID NO: 4 - DNA, 17 bp, Artificial Sequence
  Description of Artificial Sequence Synthetic primer
ggcctccgtccgagaga

SEQ ID NO: 5 - DNA, 20 bp, Artificial Sequence
  Description of Artificial Sequence Synthetic primer
caaaggaacagcagagaagc

SEQ ID NO: 6 - DNA, 24 bp, Artificial Sequence
  Description of Artificial Sequence Synthetic primer
attgagtaagacaggtccataagg

SEQ ID NO: 7 - DNA, 20 bp, Artificial Sequence
  Description of Artificial Sequence Synthetic primer
ctgtgtgcacgagggacatt

SEQ ID NO: 8 - DNA, 20 bp, Artificial Sequence
  Description of Artificial Sequence Synthetic primer
aaggctgactcggtggagaa

SEQ ID NO: 9 - DNA, 20 bp, Artificial Sequence
  Description of Artificial Sequence Synthetic primer
aaggtgaaggtcggagtcaa

SEQ ID NO: 10 - DNA, 20 bp, Artificial Sequence
  Description of Artificial Sequence Synthetic primer
aatgaaggggtcattgatgg

SEQ ID NO: 11 - DNA, 24 bp, Artificial Sequence
  Description of Artificial Sequence Synthetic primer
agaggtctcaagaaacctaggaaa

SEQ ID NO: 12 - DNA, 20 bp, Artificial Sequence
  Description of Artificial Sequence Synthetic primer
ggttcaggaccacggataga

SEQ ID NO: 13 - DNA, 22 bp, Artificial Sequence
  Description of Artificial Sequence Synthetic primer
cgagaagcttggagaatatgga

SEQ ID NO: 14 - DNA, 20 bp, Artificial Sequence
  Description of Artificial Sequence Synthetic primer
cttggtgcccactcttccta

SEQ ID NO: 15 - DNA, 22 bp, Artificial Sequence
  Description of Artificial Sequence Synthetic primer
ggatgcagaaggagattactgc

SEQ ID NO: 16 - DNA, 20 bp, Artificial Sequence
  Description of Artificial Sequence Synthetic primer
ccaccgatccacacagagta

SEQ ID NO: 17 - DNA, 19 bp, Artificial Sequence
  Description of Artificial Sequence Synthetic primer
tcgagaaccgagtgagagg

SEQ ID NO: 18 - DNA, 20 bp, Artificial Sequence
  Description of Artificial Sequence Synthetic primer
gcaaagctcctaccgtacca

SEQ ID NO: 19 - DNA, 21 bp, Artificial Sequence
  Description of Artificial Sequence Synthetic primer
aagatgctgggcaagtcgtgg

SEQ ID NO: 20 - DNA, 21 bp, Artificial Sequence
  Description of Artificial Sequence Synthetic primer
cttgtagttggggtggtcctg

SEQ ID NO: 21 - DNA, 20 bp, Artificial Sequence
  Description of Artificial Sequence Synthetic primer
cattggaaggctccctaaca

SEQ ID NO: 22 - DNA, 20 bp, Artificial Sequence
  Description of Artificial Sequence Synthetic primer
ttccactggcatcaatttca

SEQ ID NO: 23 - DNA, 18 bp, Artificial Sequence
  Description of Artificial Sequence Synthetic primer
ccatgtgctggtgtgtga

SEQ ID NO: 24 - DNA, 25 bp, Artificial Sequence
  Description of Artificial Sequence Synthetic primer
tgtgttttagttcaatgatgatcca

SEQ ID NO: 25 - DNA, 20 bp, Artificial Sequence
  Description of Artificial Sequence Synthetic primer
agatgcctcacacggagact

SEQ ID NO: 26 - DNA, 21 bp, Artificial Sequence
  Description of Artificial Sequence Synthetic primer
ggtcctctcctcctccgttcg

SEQ ID NO: 27 - DNA, 19 bp, Artificial Sequence
  Description of Artificial Sequence Synthetic primer
tcgagaaccgagtgagagg

SEQ ID NO: 28 - DNA, 19 bp, Artificial Sequence
  Description of Artificial Sequence Synthetic primer
gaaccacactcggaccaca

SEQ ID NO: 29 - DNA, 21 bp, Artificial Sequence
  Description of Artificial Sequence Synthetic primer
aagtcgcctcgaagatacaca

SEQ ID NO: 30 - DNA, 21 bp, Artificial Sequence
  Description of Artificial Sequence Synthetic primer
aaggagagaacactgctcgtg

SEQ ID NO: 31 - DNA, 22 bp, Artificial Sequence
  Description of Artificial Sequence Synthetic primer
cctcctaccttgattgcatcag

SEQ ID NO: 32 - DNA, 22 bp, Artificial Sequence
  Description of Artificial Sequence Synthetic primer
ttttgacccatagaactctgcc

SEQ ID NO: 33 - DNA, 23 bp, Artificial Sequence
  Description of Artificial Sequence Synthetic primer
ctcaatggggaggtgcagctgga

SEQ ID NO: 34 - DNA, 22 bp, Artificial Sequence
  Description of Artificial Sequence Synthetic primer
ctctcagatgatgctggtcctg

SEQ ID NO: 35 - DNA, 21 bp, Artificial Sequence
  Description of Artificial Sequence Synthetic primer
tgtgttggcttcctctgtcag

SEQ ID NO: 36 - DNA, 22 bp, Artificial Sequence
  Description of Artificial Sequence Synthetic primer
ggcagcatcctataatgagcac

SEQ ID NO: 37 - DNA, 20 bp, Artificial Sequence
  Description of Artificial Sequence Synthetic primer
aaaaggccagcgttgtctcc

SEQ ID NO: 38 - DNA, 21 bp, Artificial Sequence
  Description of Artificial Sequence Synthetic primer
tgaagccagctctctatccca

SEQ ID NO: 39 - DNA, 22 bp, Artificial Sequence
  Description of Artificial Sequence Synthetic primer
gcagcctttgtggaaccaacac

SEQ ID NO: 40 - DNA, 20 bp, Artificial Sequence
  Description of Artificial Sequence Synthetic primer
ccccgcacactaggtagaga

SEQ ID NO: 41 - DNA, 19 bp, Artificial Sequence
  Description of Artificial Sequence Synthetic primer
gacaagcgccattcacagg

SEQ ID NO: 42 - DNA, 23 bp, Artificial Sequence
  Description of Artificial Sequence Synthetic primer
tgacgtttggcaatgttattcct

(19) United States
(12) Patent Application Publication
(10) Pub. No.: US 2018/0295834 A1
(43) Pub. Date: Oct. 18, 2018

(54) CRYOPRESERVATION METHOD

(71) Applicants: The University of North Carolina at Chapel Hill, Chapel Hill, NC (US); Sapienza Universita di Roma, Roma (IT)

(72) Inventors: Lola M. Reid, Chapel Hill, NC (US); Alvaro Domenico, Rome (IT); Vincenzo Cardinale, Rome (IT); Eugenio Gaudio, Rome (IT); Guido Carpino, Rome (IT)

(73) Assignees: The University of North Carolina at Chapel Hill, Chapel Hill, NC (US); Sapienza Universita di Roma, Roma (IT)

(21) Appl. No.: 15/945,422

(22) Filed: Apr. 4, 2018

Related U.S. Application Data
(60) Provisional application No. 62/482,644, filed on Apr. 6, 2017.

(51) Int. Cl. A01N 1/02; C12N 5/071

(52) U.S. Cl. CPC A01N 1/0226; A01N 1/0221; C12N 5/0602

(57) ABSTRACT

Human biliary tree stem/progenitors (hBTSCs) are being used for cell therapies of patients with liver cirrhosis. A cryopreservation method was established to optimize sourcing of hBTSCs for these clinical programs and that comprises serum-free Kubota's Medium (KM) supplemented with 10% dimethyl sulfoxide (DMSO), ˜3% recombinant human albumin and 0.1% hyaluronans. Cryopreserved versus freshly isolated hBTSCs were similar in vitro with respect to self-replication, stemness traits, and multipotency. They were able to differentiate to functional hepatocytes, cholangiocytes or pancreatic islets, yielding similar levels of secretion of albumin or of glucose-inducible levels of insulin. Cryopreserved versus freshly isolated hBTSCs were equally able to engraft into immunocompromised mice yielding cells with human-specific gene expression and human albumin levels in murine serum that were higher for cryopreserved than for freshly isolated hBTSCs. The successful cryoypreservation of hBTSCs facilitates establishment of hBTSCs cell banking offering logistical advantages for clinical programs for treatment of liver disease.

### CROSS REFERENCE TO RELATED APPLICATIONS

This application claims priority under 35 U.S.C. 119(e) to U.S. Application No. 62/482,644, filed Apr. 6, 2017, the entirety of which is incorporated by reference herein.

### BACKGROUND

The present invention relates generally to the field of cryopreservation methods for cells.

In previous work, Applicants have demonstrated the presence of cells expressing a constellation of endodermal markers in (peri)-biliary glands of extrahepatic bile ducts^{[1-4]}. These observations in situ in human tissues have been complemented by the in vitro demonstration that subpopulations of stem cells (SOX9+/Pdx1+/Sox17+/EpCAM+; SOX9+/PDX1+/SOX17+/EpCAM−) isolated from the biliary epithelium have long-term (in vitro) maintenance and self-renewal, and are able to give rise to a more restricted progeny of different mature hepatic and pancreatic lineages^{[1-4]}. The discovery of these cells, named human biliary tree stem/progenitor cells (hBTSCs), opens a new scenario with relevant implications in different issues including the embryology of liver, biliary epithelium and pancreas, pathophysiology of biliary tree, hepatobiliary and pancreatic carcinogenesis and, finally, regenerative medicine of liver and pancreas^{[1-4]}. In this regard, the recent demonstration of the counterpart of the hBTSCs (presumed to be descendants of hBTSCs) found within the crypts of the gallbladders, named human gallbladder stem/progenitor cells (hGSCs)^{[5]}, increases the possibility of a clinical use of these populations of endodermal stem/progenitor cells with multipotential and differentiative capacity (hBTSCs and hGSCs) for cell therapies of liver diseases. Importantly, these cells are easily isolatable and cultivatable and have a low or null immunogenic and oncogenic potential^{[6]}. Given the various obstacles in cell sourcing for regenerative medicine^{[7]}, the biliary tree could represent an ideal source of stem cells and progenitors for regenerative medicine. Indeed, Applicants successfully transplanted freshly isolated hBTSCs into cirrhotic patients with benefits in terms of improvement of liver functions. Human tissues are difficult to obtain, and the current requirement for clinical programs that cells be freshly isolated hampers sourcing of cells for treatments of patients. For that reason, cryopreservation represents an obligatory step for routine uses of cell products in clinical programs of cell therapies. A number of different cryopreservation techniques have been proposed including the use of cryopreservation agents^{[8, 9]}, a cell coating technique^{[10-12]}, preconditioning techniques^{[13]}, and gradual freezing^{[14, 15]}. Unfortunately, with regard to cell types isolated from solid organs, like hepatic cells, a large variability in terms of cell viability and engraftment efficiency after thawing has been reported.^{[13,16,17]}. Terry et al.^{[18]}, for example, proposed the use of purified human serum albumin as an alternative to serum in order to preserve high viability and to achieve a defined cryopreservation condition. More recently, Turner et al.^{[19]} developed an efficient strategy to preserve adhesion molecule expression during human hepatic stem cell (hHpSC) cryopreservation by using either of two serum-free, wholly defined buffers that were supplemented with hyaluronans (HA): Crystor-10 (CS10; Biolife Solutions, Bothell, Wash., USA) or Kubota's Medium (PhoenixSongs Biologicals, Branford, Conn.).

### SUMMARY OF THE INVENTION

Aspects of the present disclosure relate to a method for cryopreservation of human biliary tree stem/progenitor cells (hBTSCs) comprising collecting human biliary tree stem/progenitor cells; adding a cryopreservation solution to the cells, in which the cryopreservation solution comprises (a) a basal medium comprising lipids, (b) hyaluronans (HA), (c) a cryoprotectant, (d) an antioxidant, and (e) a serum replacement factor, optionally albumin; and (iii) cooling the cells from an initial temperature to a final temperature at which the cells are frozen.

In some embodiments, the hyaluronan is at a concentration of between about 0.05% and 0.15%, optionally at a concentration of about 0.1%.

In some embodiments, cryoprotectant comprises one or more of sugar, glycerol, and DMSO. In some embodiments, the cryoprotectant is at a concentration of between about 1% and 20%, optionally at a concentration of about 10%.

In some embodiments, the antioxidant comprises one or more of selenium, Vitamin E, Vitamin C, and reduced glutathione.

In some embodiments, the albumin is purified albumin and/or human albumin, optionally human plasma-derived albumin or recombinant human albumin. In some embodiments, the albumin is at a concentration of between about 1 to 5%, optionally at a concentration of about 3%.

In some embodiments, the cryopreservation solution comprises one or more commercially available or otherwise disclosed buffer which may comprise one or more of components (a) through (e). Non-limiting examples include Kubota's medium, Cryostor, Viaspan, RPMI-1640, DME/F12, and GIBCO's Konckout Serum Replacement.

In some embodiments, step (iii) is accomplished using slow programmable freezing. In further embodiments, step (iii) comprises lowering the initial temperature at a rate of about 1° C. per minute until a final temperature is reached. In some embodiments, step (iii) comprises: (a) cooling cells from an initial temperature to a final temperature of about −80° C. using solid carbon dioxide, or (b) cooling cells from an initial temperature to a final temperature of about −196° C. using liquid nitrogen. It is appreciated that step (iii) may be accomplished, in certain embodiments, using the rapid freezing methods disclosed herein.

Further aspects relate to a method of thawing of the cryopreserved human biliary tree stem/progenitor cells (hBTSCs) disclosed herein. Non-limiting examples of suitable thawing, e.g. (i) thawing cells cryopreserved according to the method disclosed herein, (ii) adding a first buffer solution; (iii) separating the cells from the cryopreservation medium and the first buffer solution; and (iv) resuspending the cells in a second buffer solution.

In some embodiments the first and/or second buffer solution comprise serum or a serum replacement medium. In some embodiments, the serum is fetal bovine serum. In some embodiments, the serum replacement medium may be one or more of GIBCO's Knockout Serum Replacement Medium and Kubota's medium, optionally supplemented with albumin, which in turn is optionally human serum-derived albumin. In some embodiments, the serum is at a concentration of between about 2% to 20%, optionally between about 10% to 20%, about 10%, or about 20%. It is appreciated that this “high serum” thawing method may be advantageous to minimize ice crystal formation where a non-isotonic buffer is used because of the need for high lipid content in this process. In some embodiments, the serum is at a concentration of between about 2% to 5%. It is appreciated that this “low serum” thawing method may be used where an isotonic buffer is used because high lipid content is not required. In some embodiments, the serum replacement medium comprises albumin at a concentration of between about 1% to 5%.

In some embodiments, the first and/or second buffer solution comprise a thawing buffer. It is appreciated that some commercially available thawing buffers comprise serum or a serum replacement. It is also appreciated that some embodiments may include thawing through means other than those prescribed herein above.

It is further appreciated that multiple ways exist to separate cells from a medium, e.g. culture medium, buffer solution, and/or cryopreservation solution. Non-limiting examples include centrifuging the cells; filtration of the cells through a sieve or filter; and French-press type filtration.

Additional aspects relate to a method of culturing thawed, cryopreserved human biliary tree stem/progenitor cells comprising plating the cells thawed according to the method disclosed herein; culturing the cells in an incubator; removing the buffer solution; and replacing the buffer solution with a culture medium designed for the growth and/or differentiation of human biliary tree stem/progenitor cells.

In some embodiments, the cells are incubated in the incubator for between about 6 to 7 hours.

In some embodiments, the culture medium designed for the growth and/or differentiation of human biliary tree stem/progenitor cells comprises Kubota's medium and/or a hormonally defined medium (HDM) for the differentiation of cells (e.g. for lineage restriction to hepatocytes, then. HDM-H).

Further aspects relate to a composition comprising a plurality of cryopreserved human biliary tree stem/progenitor cells according to the methods disclosed herein. In some embodiments, these cells may be thawed or frozen.

### BRIEF DESCRIPTION OF THE DRAWINGS

FIGS. 1A-1E depicts biological cell functions after cryopreservation/thawing. A) Cell viability was assessed by Trypan blue exclusion test after thawing the cells cryopreserved in different solutions (N=9 experiments). Viability was significantly higher in solution 1 (Sol1) and Sol3 vs Sol2A, Sol2B, control solution (CTRL), and freshly isolated (No Cryo). No difference was found between Sol1 and Sol3. Data are expressed as mean±SD of 9 experiments; [symbol] =p<0.001 Sol1 and Sol3 vs Sol2A, Sol2B, and CTRL; [symbol] =p<0.001 No Cryo vs all other Solution. Solution composition: Sol1=Kubota Medium (KM), DMSO (10%), recombinant human albumin (15%), hyaluronic acid (0.1% W/V); Sol2A=KM, hyaluronic acid (0.1% W/V), DMSO (10%); Sol2B=KM, hyaluronic acid (0.05% W/V), DMSO (10%); Sol3=KM, DMSO (10%), recombinant human albumin (15%); CTRL=KM, DMSO (10%), recombinant human albumin (1.5%). B) Cell senescence was evaluated by X-Gal test in cultures obtained from cryopreserved or freshly isolated cells (No Cryo) obtained from the same donors. Graphics show the percentage of X-Gal negative cells (non senescent cells). X-Gal negative cells exceeded 95% after cryopreservation. No difference was observed between Sol1 and Sol3, and among cryopreserved cells and fresh control cells (No Cryo). Sol2A demonstrated a massive senescence of cultured thawed cells (δ=p<0.0001 vs others). Data are expressed as mean±SD of 3 experiments. C) Proliferation rate expressed as population doubling (PD) week rate in cultures of hBTSCs cryopreserved in Sol1, Sol3, and freshly isolated controls (No Cryo). Cryopreserved cells (Sol1 and Sol3) demonstrated a higher PD week rate with respect non-cryopreserved cells (§ =p<0.01). Data are expressed as mean±SD of 8 experiments. D) Population Doubling Time (PDT) appeared lower in Sol1 (with hyaluronic acid/HA) than Sol3 (without HA) and freshly isolated controls (No Cryo) ( [symbol] =p<0.001 vs others), and in Sol3 vs freshly isolated controls (No Cryo) (δ=p<0.0001 vs No Cryo). Data are expressed as mean±SD of 8 experiments. E) The number of colonies was counted at day 3 of culture. HA-coated hBTSCs and uncoated hBTSCs were compared. Graphics illustrate the number of colony formed after thawing cells cryopreserved in Sol1 and Sol3. A higher number of colonies (31.56±8.43) developed in cultures from Sol1 than Sol3 (10.11±3.85) ($=p<0.000001). Data are expressed as mean±SD of 18 experiments.

FIG. 2 shows expression of pluripotency and molecule adhesion genes in cultures from cryopreserved cells in solution 1 (Sol1), Sol3, or freshly isolated, that is not cryopreserved (No Cryo) human biliary tree stem cells (hBTSCs). Relative gene expression of SOX2. Cryopreserved hBTSCs in both Sol1 and 3 showed increased expression. Data are expressed as mean±standard error (SE) of 9 experiments; *=p<0.05. Relative gene expression of PDX1. Cryopreserved hBTSCs in both Sol1 and 3 showed increased expression. Data are expressed as mean±SE of 9 experiments; *=p<0.05. Relative gene expression of NANOG. Cryopreserved hBTSCs in both Sol1 and 3 showed increased expression. Data are expressed as mean±SE of 9 experiments; § =p<0.01. Relative gene expression of SOX17. Cryopreserved hBTSCs in both Sol1 and 3 showed increased expression. Data are expressed as mean±SE of 9 experiments; *=p<0.05. Relative gene expression of OCT4. Cryopreserved hBTSCs in both Sol1 and 3 showed increased expression. Data are expressed as mean±SE of 9 experiments; § =p<0.01. Relative gene expression of CD44. Data are expressed as mean±standard error (SE) of 6 experiments. Relative gene expression of ITGβ1. Cryopreserved hBTSCs in both Sol1 and 3 showed reduced expression. Data are expressed as mean±SE of 6 experiments; *=p<0.05. Relative gene expression of ITGPβ4. Cryopreserved hBTSCs in both Sol1 and 3 showed increased expression. Data are expressed as mean±SE of 6 experiments; *=p<0.05 No Cryo vs others. Relative gene expression of CDH1. Cryopreserved hBTSCs in both Sol1 and 3 showed reduced expression. Data are expressed as mean±SE of 6 experiments; § =p<0.01.

FIGS. 3A-3B shows expression of pluripotency and multipotency genes in cultures of cryopreserved or freshly isolated hBTSCs under self renewal (KM) or hormonally defined medium for multiple endodermal mature fates (hepatocytic/HM, cholangiocytic/CM, pancreatic islets/PM). A) Relative gene expression of SOX2, EpCAM, OCT4, PDX1, SOX17, SOX2 in cryopreserved hBTSCs in Sol1 and in Sol3 (not shown) under different culture conditions. Previously cryopreserved hBTSCs cultured under self-renewal conditions in Kubota's Medium (KM) reduced the expression of pluripotency and multipotency genes when transferred in hormonally defined medium for particular endodermal mature fates (hepatocytic/HM, cholangiocytic/CM, pancreatic islets/PM). Data are expressed as mean±SD of 3 experiments; *=p<0.05; § =p<0.01; **=p<0.05 HM vs CM and PM; §§ =p<0.05 PM vs CM and HM. B) Relative gene expression of Nanog, SOX2, EpCAM, OCT4, PDX1, SOX17, SOX2 in freshly isolated (FI) hBTSCs cultured in different defined conditions. Freshly isolated hBTSCs cultured under self-renewal conditions in Kubota's Medium (KM) reduced the expression of pluripotency and multipotency genes when transferred in hormonally defined medium for particular endodermal mature fates (hepatocytic/HM, cholangiocytic/CM, pancreatic islets/PM). Data are expressed as mean±SD of 3 experiments; *=p<0.05; § =p<0.01**=p<0.05 HM vs CM and PM; §§ =p<0.05 PM vs CM and HM.

FIGS. 4A-4B shows expression of specific mature fate genes in cultures of cryopreserved or freshly isolated hBTSCs in self-renewal conditions (Kubota's Medium-KM) or hormonally defined medium for particular endodermal mature fates (hepatoytic/HM, cholangiocytic/CM, pancreatic islets/PM). A) Relative gene expression of CYP3A4, albumin (ALB), transferrin (TRANSF), insulin (INS), glucagon, Secretin Receptor (SR), CFTR, ASBT in cryopreserved hBTSCs cultured in different defined conditions. Previously cryopreserved hBTSCs cultured in self-renewal conditions in Kubota's Medium (KM) increased the expression of specific genes associated with adult fates when transferred in the appropriate hormonally defined medium (hepatocytic/HM, cholangioytic/CM, pancreatic islets/PM). Data are expressed as mean±SD of 3 experiments; *=p<0.05; § =p<0.01; [symbol] =p<0.001; δ=p<0.0001. B) Relative gene expression of CYP3A4, albumin (ALB), transferrin (TRANSF), insulin (INS), glucagon, Secretin Receptor (SR), CFTR, ASBT in freshly isolated hBTSCs cultured in different defined conditions. Freshly isolated hBTSCs cultured in self-renewal conditions in Kubota's Medium (KM) increased the expression of specific genes associated with mature fates when transferred in the related hormonally defined medium (hepatocytic/HM, cholangiocytic/CM, pancreatic islets/PM). Data are expressed as mean±SD of 3 experiments; *=p<0.05; § =p<0.01; [symbol] =p<0.001; δ=p<0.0001.

FIGS. 5A-5B depicts morphological, phenotypic and functional changes induced by hormonally defined culture media compared to Kubota's Medium/KM (basal condition) to demonstrate the effective differentiation of cryopreserved hBTSCs. A) Cryopreserved hBTSCs were thawed and then cultured in media specifically tailored to induce differentiation in hepatocytes (HM), cholangiocyetes (CM) or pancreatic cells (PM). After 15 days in HM, cuboidal-shaped cells expressing albumin (hepatocyte markers) were evident (N=5). After 15 days in CM, clusters of cells expressing CK19 appeared (N=5). After 14 days, the monolayers in PM transition to dense balls of aggregated cells budding from the edges of the colonies and containing cells expressing insulin (FIG. 10) (N=5). Figures are representative of cultures of cells cryopreserved in Sol1 (N=5). B) The differentiation of cryopreserved hBTSCs thawed and cultured in hepatocytic medium (HM) was demonstrated by the albumin secretion with respect to control cells cultured in self-renewal conditions in Kubota's Medium (KM) (data are expressed as mean±SD of 6 experiments; § =p<0.01 HM vs KM), that resulted lower with respect to HepG2 (*=p<0.05 HepG2 vs KM), but similar to freshly isolated cells (not shown). C) In pancreatic medium (PM), both cryopreserved and freshly isolated hBTSCs acquired insulin (C-Peptide) secretion property that was regulated by glucose concentration (data are expressed as mean±SD of 7 experiments; § =p<0.01 low vs high glucose concentration, [symbol] =p<0.001 low vs high glucose concentration).

FIGS. 6A-6C depicts in vivo liver engraftment and hepatocyte differentiation of hBTSCs (cryopreserved vs freshly isolated) after intrasplenic transplantation in SCID mice. Thirty days after hBTSC injection into the spleen, livers and serum were analyzed. A) Sections of livers were analyzed by immunohistochemistry utilizing anti-human mitochondria. Freshly isolated and cryopreserved hBTSCs showed similar engraftment efficiency into the murine liver parenchyma (N=3). The expression of human mitochondria in liver parenchyma of SCID mice indicated that 2.626±1.530% and 3.722±0.639% of the host's parenchyma cell mass derived from transplanted freshly isolated and cryopreserved hBTSCs respectively (data are expressed as the mean±SD of 3 experiments). B) Sections of livers were analyzed by RT-qPCR for human albumin gene expression. The gene expression of human albumin in liver parenchyma of SCID mice was higher (§ =p<0.01) when cryopreserved hBTSCs (5.19*10⁻⁷+3.06*10⁻⁷) were transplanted as compared to when freshly isolated hBTSCs (1.90*10⁻¹⁰+1.09*10⁻¹⁰) were transplanted. (Data are expressed as the mean±SD of 3 experiments). C) levels of human serum albumin in the SCID mice were significantly higher (δ=p<0.0001) when cryopreserved hBTSCs (76.39±17.04 ng/mL) were transplanted with respect to freshly isolated hBTSCs (24.13±1.44 ng/mL). (Data are expressed as mean±SD of 3 experiments).

FIGS. 7A-7D depicts single cell clonogenity via contrast phase imagines (Magnifications 10×) of a single colony at different culture times. A) Day 1, B) Day 3, C) Day 7, D) Day 10.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting. All references mentioned herein and throughout the application are incorporated by reference.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker ed (1987) Immunochemical Methods in Cell and Molecular Biology (Academic Press, London); and Herzenberg et al. eds (1996) Weir's Handbook of Experimental Immunology.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

### Definitions

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration (e.g., the percentage of collagen in the total proteins in the biomatrix scaffold) and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

The terms “buffer” and/or “rinse media” are used herein to refer to the reagents used in the preparation of the biomatrix scaffolds.

As used herein, the term “cell” refers to a eukaryotic cell. In some embodiments, this cell is of animal origin and can be a stem cell or a somatic cell. The term “population of cells” refers to a group of one or more cells of the same or different cell type with the same or different origin. In some embodiments, this population of cells may be derived from a cell line; in some embodiments, this population of cells may be derived from a sample of an organ or tissue.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The term “culture” or “cell culture” means the maintenance of cells in an artificial, in vitro or ex vivo two dimensional (2D, monolayer) or three dimensional (3D) environment (polarized shapes of cells when on certain forms of matrix or when floating), in some embodiments as adherent cells (e.g. monolayer cultures) or as floating aggregates cultures of spheroids or organoids. The term “spheroid” indicates a floating aggregate of cells all being the same cell type (e.g. an aggregate from a cell line); an “organoid” is a floating aggregate of cells comprised of multiple cell types. In some embodiments, the organoid may be an aggregate of epithelia and one or more mesenchymal cell types comprising endothelia and/or stromal or stellate cells. A “cell culture system” is used herein to refer to culture conditions in which a population of cells may survive or be grown.

“Culture medium” is used herein to refer to a nutrient solution for the culturing, growth, or proliferation of cells. In some embodiments, it comprises one or more of amino acids, vitamins, salts, lipids, minerals, trace elements) and mimicking the chemical constituents of interstitial fluid. Culture medium may be characterized by functional properties such as, but not limited to, the ability to maintain cells in a particular state (e.g. a pluripotent state, a quiescent state, etc.), to mature cells—in some instances, specifically, to promote the differentiation of stem/progenitor cells into cells of a particular lineage. A non-limiting example of culture medium used for stem/progenitors is Kubota's Medium, which is further defined herein below. In some embodiments the medium may be a “seeding medium” used to present or introduce cells into a given environment.

More specifically, a “basal medium” is a buffer comprised of amino acids, sugars, lipids, vitamins, minerals, salts, trace elements and various nutrients in compositions that mimic the chemical constituents of interstitial fluid around cells. Such media may optionally be supplemented with serum to provide requisite signaling molecules (hormones, growth factors) needed to drive a biological process (e.g. proliferation, differentiation) or as a source of inhibitors to enzymes used typically in the preparation of cell suspensions. Although the serum can be autologous to the cell types used in cultures, it is most commonly serum from animals routinely slaughtered for agricultural or food purposes such as serum from cows, sheep, goats, horses, etc. Media supplemented with serum may be optionally referred to as serum supplemented media (SSM).

As used herein, “differentiation” means that specific conditions cause cells to mature to adult cell types that produce adult specific gene products.

The terms “equivalent” or “biological equivalent” are used interchangeably when referring to a particular molecule, biological, or cellular material and intend those having minimal homology while still maintaining desired structure or functionality.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

The term “gene” as used herein is meant broadly to include any nucleic acid sequence transcribed into an RNA molecule, whether the RNA is coding (e.g., mRNA) or non-coding (e.g., ncRNA).

As used herein, the term “generate” and its equivalents (e.g. generating, generated, etc.) are used interchangeably with “produce” and its equivalents when referring to the method steps that yield a particular model colony, organ, or organoid.

The term “isolated” as used herein refers to molecules or biologicals or cellular materials being substantially free from other materials.

“Kubota's Medium” as used herein refers to a serum-free, wholly defined medium designed for endodermal stem cells and enabling them to expand clonogenically in a self-replicative mode of division (especially if on hyaluronan substrata or in 3D, if hyaluronans are added to the medium). Kubota's medium may refer to any basal medium containing no copper, low calcium (<0.5 mM), insulin, transferrin/Fe, a mix of purified free fatty acids bound to purified albumin and, optionally, also high density lipoprotein. Kubota's Medium or its equivalent is used serum-free, especially in culture selection for endodermal stem cells, and contains only a defined mix of purified signals (insulin, transferrin/Fe), lipids, and nutrients. In some embodiments, it can be used transiently as a SSM using low (typically 5% or less) levels of serum for the seeding process of introducing cells into the matrix scaffolds and in order to inactivate enzymes used in preparing cell suspensions; switching to the serum-free Kubota's Medium as quickly as possible (e.g. within 5-6 hours) is optimal.

In certain embodiments, the medium is comprised of a serum-free basal medium (e.g., RPMI 1640 or DME/F12) containing no copper, low calcium (<0.5 mM) and supplemented with insulin (5 μg/mL), transferrin/Fe (5 μg/mL), high density lipoprotein (10 μg/mL), selenium (10⁻¹⁰ M), zinc (10⁻¹² M), nicotinamide (5 μg/mL), and a mixture of purified free fatty acids bound to a form of purified albumin. Non-limiting, exemplary methods for the preparation of this media have been published elsewhere, e.g., Kubota H, Reid L M, Proceedings of the National Academy of Sciences (USA) 2000; 97:12132-12137, Y. Wang, H. L. Yao, C. B. Cui et al. Hepatology. 2010; 52(4):1443-54, Turner et al; Journal of Biomedical Biomaterials. 2000; 82(1): pp. 156-168; Y. Wang, H. L. Yao, C. B. Cui et al. Hepatology. 2010 October 52(4):1443-54, the disclosures of which is incorporated herein by reference. Variants of Kubota's Medium can be used for certain cell types by providing additional factors and supplements to allow for expansion under serum free conditions. For example, Kubota's Medium may be modified to enable transit amplifying cells or committed progenitors (e.g. hepatoblasts) and other maturational lineage stages later than stem cell populations to survive and expand ex vivo under serum-free conditions. One example of this is Kubota's Medium modified for ex vivo expansion of hepatoblasts and their descendants, committed progenitors: serum-free Kubota's Medium is further supplemented with hepatocyte growth factor (HGF), epidermal growth factor (EGF), basic fibroblast growth factor (bFGF), and sometimes vascular endothelial growth factor (VEGF). The resulting cell expansion occurs with minimal (if any) self-replication. The medium is especially effective if the cells are on substrata of type IV collagen and laminin or embedded in 3-D hydrogels containing more than 50% type IV collagen and laminin.

The terms “nucleic acid,” “polynucleotide,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers.

A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

### Abbreviations

The following abbreviations are used in the examples disclosed below.

ALB, Albumin; ASBT, Apical Sodium dependent Bile acid Transporter; bFGF, basic Fibroblast Growth Factor; CDH1, Cadherin 1; CFTR, Cystic Fibrosis Transmembrane conductance Regulator; CK, Cytokeratin; CYP3A4, Cytochrome P450 3A4; DMSO, Dimethyl-Sulfoxide; DPBS, Dulbecco's Phosphate-Buffer Saline; EGF, Epidermal Growth Factor; EpCAM, Epithelial Cell Adhesion Molecule; FBS, Fetal Bovine Serum; GAPDH, Glyceraldehyde 3-Phosphote Dehydrogenase; GMP, Good Manufacturing Pratice; HA, Hyaluronans; hBTSCs, human Biliary Tree Stem/progenitor Cells; HGF, Hepatocyte Growth Factor; hGSCs, human Gallbladder Stem/progenitor Cells: hHpSC, human hepatic stem cells; HDM, serum-free, hormonally defined medium; HSA, Human Serum Albumin; INS, Insulin; ITGB1, Integrin β1; ITGB4, Integrin β 4; KM, Kubota's Medium; MKM, Modified Kubota's Medium; NANOG, Nanog homeobox; OCT4, Octamer-binding Transcription factor 4; OSM, oncostatin M; PBG, peri-biliary gland; PD, Population Doubling; PDT, Population Doubling Time; PDX1, pancreatic and duodenal homeobox 1; RT-qPCR, Quantitative Reverse-Transcription Polymerase Chain Reaction; SCID, Severe Combined Immunodeficiency; SD, Standard Deviation; SOX, Sry-related HMG box; SR, Secretin Receptor; T3, Triidrothyroxine 3; TRANSF, Transferrin; VEGF, Vascular Endothelia Growth Factor.

### Modes of Carrying Out the Disclosure

In general, techniques of cryopreservation rely on the use of isotonic buffers. Such buffers are known to have the least propensity to generate ice crystal formation, since there are no shifts of water due to osmotic effects.

An example of this is Cryostor, a cryopreservation buffer sold by Biolife Solutions and being a derivative of the University of Wisconsin organ preservation buffer. The base buffer is isotonic and is supplemented with an antifreeze protein (such as found in animals that live in the artic)+a cryopreservative (DMSO)+a sugar (a specific size of dextran).

Applicants discovered that the use of a non-isotonic buffer could also be appropriate for cryopreservation. For example, Kubota's Medium is not isotonic, but the osmotic effects are alleviated by the hyaluronans. The hyaluronans form a complex with the surface receptors for adhesion molecules and block their internalization. Therefore, when the cells are thawed, they are able to attach immediately. Moreover, Applicants have demonstrated that it is ideal for the stem cells in that they do not have to be switched from one type of buffer to another. Rather they are kept in the same medium with use of the supplements to minimize any osmotic effects. Indeed, if cells are cryopreserved in Kubota's Medium, they may be thawed in it and plated, enabling users to avoid the centrifugation step (e.g. eliminating worry about having DMSO in the medium for the few hours during attachment, and simply letting the cells attach and then gently removing the medium after a few hours. The medium may then be replaced with fresh serum-free Kubota's Medium). Aspects relating to the use of Kubota's medium in cryopreservation are disclosed in PCT/US2011/035498, incorporated by reference herein.

In general, all cryopreservation buffers use a cryopreservative such as DMSO. Natural cryopreservatives include sugar (e.g. glucose) or glycerol; these are naturally occurring cryopreservatives in a number of animal species. Although glycerol may be used, it is quite viscous. In the past, investigators have found that DMSO has a tendency to be more soluble and easier to use. Some cryopreservation buffers add an antifreezing protein derived from animals that are found in arctic climates. These proteins have been characterized and cloned enabling their availability commercially.

Many cryopreservative buffers use an antioxidant. Non-limiting examples include selenium, vitamin E, and vitamin C.

Slow-freezing and rapid cryopreservation techniques are known in the art. For rapid cryopreservation, a typical method is to add the cells to the cryopreservative buffer, pack the ampoule or container in cotton, and put it into a −80° C. freezer. The viabilities of the cells are not as good (e.g. around 60-70%) as with slow freezing but for some purposes, this cruder method is acceptable. For optimal freezing, to achieve cell viabilities on thawing above 80-90%, one must use slow freezing methods. There are multiple forms of computerized freezing chambers that will reduce the temperature a degree at a time until it reaches −80° C.; they often include a computerized strategy of having the cells linger for somewhat longer at a temperature at which ice begins to form to minimize ice crystal damage to the cells.

When cryopreserving stem cells, there needs to be high levels of lipids in the buffer. Applicants achieved this using Kubota's Medium that is replete with free fatty acids complexed with purified albumin. Another medium, GIBCO's Knockout Serum Replacement Medium, similarly uses a lot of lipids, and is known to be employed for cryopreserving ES cells and iPS cells.

Applicants further discovered that use of higher levels (levels nearing those in vivo) of highly purified, recombinant human albumin during cryopreservation yielded unexpectedly superior results.

Aspects of the present disclosure relate to a method for cryopreservation of human biliary tree stem/progenitor cells (hBTSCs) comprising collecting human biliary tree stem/progenitor cells; adding a cryopreservation solution to the cells, in which the cryopreservation solution comprises (a) a basal medium containing lipids, (b) hyaluronans (HA), (c) a cryoprotectant, (d) an antioxidant, and (e) a serum replacement factor, optionally albumin; and (iii) cooling the cells from an initial temperature to a final temperature at which the cells are frozen.

In some embodiments, the hyaluronan is at a concentration of between about 0.05% and 0.15%, optionally at a concentration of about 0.1%.

In some embodiments, cryoprotectant comprises one or more of sugar, glycerol, and DMSO. In some embodiments, the cryoprotectant is at a concentration of between about 1% and 20%, optionally at a concentration of about 10%.

In some embodiments, the antioxidant comprises one or more of selenium, Vitamin E, Vitamin C, and reduced glutathione.

In some embodiments, the albumin is purified albumin and/or human albumin, optionally human plasma-derived albumin or recombinant human albumin. In some embodiments, the albumin is at a concentration of between about 1 to 5%, optionally at a concentration of about 3%, mimicking the known concentration of albumin in serum (3-5%).

In some embodiments, the cryopreservation solution comprises one or more commercially available or otherwise disclosed buffers which may comprise one or more of components (a) through (e). Non-limiting examples include Kubota's medium, Cryostor, RPMI-1640, DME/F12, and GIBCO's Konckout Serum Replacement.

In some embodiments, step (iii) is accomplished using slow programmable freezing. In further embodiments, step (iii) comprises lowering the initial temperature at a rate of about 1° C. per minute until a final temperature is reached. In some embodiments, step (iii) comprises: (a) cooling cells from an initial temperature to a final temperature of about −80° C. using solid carbon dioxide, or (b) cooling cells from an initial temperature to a final temperature of about −196° C. using liquid nitrogen. It is appreciated that step (iii) may be accomplished, in certain embodiments, using the rapid freezing methods disclosed herein.

Further aspects relate to a method of thawing of the cryopreserved human biliary tree stem/progenitor cells (hBTSCs) disclosed herein. Non-limiting examples of suitable thawing, e.g. (i) thawing cells cryopreserved according to the method disclosed herein, (ii) adding a first buffer solution; (iii) separating the cells from the cryopreservation medium and the first buffer solution; and (iv) resuspending the cells in a second buffer solution.

In some embodiments the first and/or second buffer solution comprise serum or a serum replacement medium. In some embodiments, the serum is fetal bovine serum. In some embodiments, the serum replacement medium may be one or more of GIBCO's Knockout Serum Replacement Medium and Kubota's medium, optionally supplemented with albumin, which in turn is optionally human serum-derived albumin. In some embodiments, the serum is at a concentration of between about 2% to 20%, optionally between about 10% to 20%, about 10%, or about 20%. It is appreciated that this “high serum” thawing method may be advantageous to minimize ice crystal formation where a non-isotonic buffer is used because of the need for high lipid content in this process. In some embodiments, the serum is at a concentration of between about 2% to 5%. It is appreciated that this “low serum” thawing method may be used where an isotonic buffer is used because high lipid content is not required. In some embodiments, the serum replacement medium comprises albumin at a concentration of between about 1% to 5%.

In some embodiments, the first and/or second buffer solution comprise thawing buffer. It is appreciated that some commercially available thawing buffers comprise serum or serum replacement. It is also appreciated that some embodiments may include thawing through means other than those prescribed herein above.

It is further appreciated that multiple ways exist to separate cells from supernatant, e.g. culture medium, buffer solution, and/or cryopreservation solution. Non-limiting examples include centrifuging the cells; filtration of the cells through a sieve or filter; and French-press type filtration.

Additional aspects relate to a method of culturing thawed, cryopreserved human biliary tree stem/progenitor cells comprising plating the cells thawed according to the method disclosed herein; culturing the cells in an incubator; removing the buffer solution; and replacing the buffer solution with a culture medium designed for the growth and/or differentiation of human biliary tree stem/progenitor cells.

In some embodiments, the cells are incubated in the incubator for between about 6 to 7 hours.

In some embodiments, the culture medium designed for the growth and/or differentiation of human biliary tree stem/progenitor cells comprises Kubota's medium and/or a hormonally defined medium (HDM) for the differentiation of cells (e.g. for lineage restriction to hepatocytes, then. HDM-H).

Further aspects relate to a composition comprising a plurality of cryopreserved human biliary tree stem/progenitor cells according to the methods disclosed herein. In some embodiments, these cells may be thawed or frozen.

### EXAMPLES

The following examples are non-limiting and illustrative of procedures which can be used in various instances in carrying the disclosure into effect. Additionally, all reference disclosed herein below are incorporated by reference in their entirety.

### Example 1—Cryopreservation Studies

I. Materials and Methods

### Human Tissue Sourcing.

For in vitro experiments, human extrahepatic biliary tree, comprising common hepatic duct, bile duct, cystic duct, gallbladder, and hepato-pancreatic ampulla were obtained from organ donors from the “Paride Stefanini” Department of General Surgery and Organ Transplantation, Sapienza University of Rome, Rome, Italy. Informed consent to use tissues for research purposes was obtained from our transplant program. All samples derived from adults between the ages of 19 and 73 years. For in vivo experiments, hBTSCs isolated from fetal livers have been utilized. Human fetuses (16-22-week gestational age) were obtained by elective pregnancy termination from the Department of Gynecology (Sapienza, University of Rome, Italy). Informed consent was obtained from the mother before abortion. The study was approved by the local ethics committee of the Sapienza University Hospital. Protocols received the approval of our Institutional Review Board, and processing was compliant with current Good Manufacturing Practice (cGMP). The research protocol was reviewed and approved by the Ethic Committee of Umberto I University Hospital, Rome.

### Tissue Processing.

Tissue specimens were processed as previously described 1, 5, 6, 28-30. In brief, tissues were digested in GMP Serum-free Dendritic Cell Medium (CellGro #20801-0500) supplemented with 0.1% Octalbin 20% (Octapharma #5400454), 1 nM selenium, antibiotics, 300 U/ml Collagenase NB1 GMP (Serva #17452.01), 100 U/ml Pulmozyme (Roche #18450.02), at 37° C. with frequent agitation for 30-45 min. Suspensions were filtered through a 800 micron metallic mesh filter (IDEALE ACLRI9 inox stainless steel) and spun at 270 g for 10 min before resuspension. Thereafter, cell suspensions were passed consecutively through a 100 and 30 micron (μ) mesh filter; then, cell counting was done by Fast-Read 102 (BiosigmaSrl, Venice, Italy) and cell viability by the Trypan Blue assay measured (expressed as % of viable cells over total cells). Cell viability (trypan blue exclusion) was consistently higher than 95%.

### EpCAM Sorting Procedures.

Cells were sorted for expression of Epithelial cell adhesion molecule (EpCAM) by using magnetic beads as indicated by the manufacturer (MiltenyiBiotec Inc., Germany). Briefly, the EpCAM+ cells were magnetically labelled with EpCAM MicroBeads (MiltenyiBiotec Inc., catalog #130-061-101). Then, the cell suspension was loaded onto a MACS LS Column (Miltenyi Biotec Inc., catalog #130-042-401) that was placed in the magnetic field of a MACS Separator. EpCAM+ cells were suspended in basal medium at a concentration of 300,000 cells per mL, and used as the final cell suspension.

### Cell Isolation in GMP Conditions and Sterility Testing.

To produce hBTSCs in cGMP conditions for future clinical application, gallbladders were processed following “The rules governing medicinal products in the European Union” and the European guidelines of good manufacturing practices for medicinal products for human use (EudraLex—Volume 4 Good manufacturing practice Guidelines).

### Media and Solutions.

All media were sterile-filtered (0.22-μm filter) and kept in the dark at 4° C. before use. RPMI-1640, the basal medium used for all the cell cultures, and fetal bovine serum (FBS) were obtained from GIBCO/Invitrogen (Carlsbad, Calif.). All reagents were obtained from Sigma (St. Louis, Mo.) unless otherwise specified. Growth factors, except those noted, were purchased from R&D Systems (Minneapolis, Minn.).

Kubota's Medium (KM) is a serum-free medium developed for survival and expansion of endodermal stem/progenitors³¹ and subsequently shown to be successful with human hepatic stem cells^{28, 29}, human biliary tree stem cells^{1, 3, 4}, human pancreatic stem/progenitor cells²⁵ and rodent hepatic stem cells³². It consists of any basal medium (here being RPMI 1640) with no copper, low calcium (0.3 mM), 10⁻⁹ M Selenium, 4.5 mM Nicotinamide, 0.1 nM Zinc Sulphate heptahydrate, 10⁻⁸ M hydrocortisone (or dexamethasone), 5 μg/mL transferrin/Fe, 5 μg/mL insulin, 10 μg/mL high density lipoprotein, 0.1% human (or bovine) serum albumin (HSA or BSA), and a mixture of purified free fatty acids that are added bound to purified HSA. The detailed protocol of its preparation was first reported by Kubota and Reid³¹ and subsequently summarized in various reviews²⁸. It is now available commercially through PhoenixSongs Biologicals (Branford, Conn.).

For differentiation studies, serum-free Kubota's Medium was supplemented with calcium (final concentration 0.6 mM), copper (10⁻¹² M) and 20 ng/mL basic fibroblast growth factor (bFGF) and referred to as modified Kubota's Medium (MKM). Three different HDM have been prepared to induce selective differentiation of hBTSCs:
HDM for Hepatocyte differentiation (HM): was prepared supplementing MKM with 7 μg/L glucagon, 2 g/L galactose, 1 nM triiodothyroxine 3 (T3), 10 ng/mL Oncostatin M (OSM); 10 ng/mL epidermal growth factor (EGF), 20 ng/mL hepatocyte growth factor (HGF), and 1 μM dexamethasone^{4, 6}
HDM for Cholangiocyte differentiation (CM): MKM supplemented with 20 ng/mL vascular endothelial cell growth factor (VEGF) 165 and 10 ng/mL HGF^{4, 6}
HDM for Pancreatic islet cell differentiation (PM): MKM without hydrocortisone, supplemented with 2% B27, 0.1 mM ascorbic acid, 0.25 μM cyclopamine, 1 μM retinoic acid; bFGF was added for the first 4 days and then replaced with 50 ng/mL exendin-4 and 20 ng/mL of HGF^{4, 5}.

### Methods and Buffers for Cryopreservation

The cells were detached from the various plastic substrata to be collected and cryopreserved. Detached cell cultures were centrifuged at 270 g for 10 minutes, and 1 mL of the solution of cryopreservation was added to the cell pellets. Finally the buffers containing the cells were transferred into Nunc vials (Unimed #6302598). These were placed into Nalgene Cryo 1° C. Freezing Container (Nalgene, CAT No. 5100-0001). The method of cryopreservation used was by lowering of the temperature at 1° C. per minute to −80° C.; after 24 hours, the cells were placed in liquid nitrogen at −196° C.

Different candidate cryopreservation buffers were tested. They were prepared on the day of use and in the amount of 10 mL each. The buffers are derivative of those established by Turner, et al¹⁹. They all consist of Kubota's Medium, a serum-free medium developed for endodermal stem/progenitors and supplemented with 10% DMSO; in addition, KM contains purified albumin to which is bound a mix of purified free fatty acids. In some of the buffers, additional, higher levels of albumin were added. The albumin is prepared from recombinant human albumin solutions (Octalbin 20%; Octapharma #5400454). Thus, the 15% solution is 15% of the 20% Octalbin preparation or a final percentage of 3%; the 1.5% is, therefore, 0.3%. The distinctions among the buffers are as follows:
Sol1: recombinant human albumin (15%), HA (0.1%)
Sol2A: HA (0.1%)
Sol2B: HA (0.05%),
Sol3: recombinant human albumin (15%),
CTRL: recombinant human albumin (1.5%),

HA was prepared using 200 mg of sodium hyaluronate suspended in 30 mL of KM.

### Cell Thawing.

The frozen cells in the Nunc (Unimed #6302598) were thawed and 1 mL of culture medium with 20% Human Serum-derived Albumin was added slowly (drop by drop). Then, the contents were transferred into a 15 mL Falcon tube; the volume was brought slowly to 5 mL with KM and then subjected to centrifugation at 270 g for 10 minutes². After centrifugation the supernatant was removed, eliminating the DMSO that was used for the cryopreservation. The cell pellet was resuspended to the requisite volume for plating with KM supplemented with 10% serum¹. Analytical studies included ones assessing the cell viability of thawed cells that had been frozen with the different cryopreservation solutions, and gene expression, through the use of RT-qPCR, both of adhesion molecules (ITGB1, ITGB4, CD44, CDH1) that are markers of pluripotent stem cells and markers of endodermal stem cells (PDX1, OCT4, SOX17, SOX2, Nanog).

### Cell Cultures and Clonal Expansion.

Unsorted and sorted EpCAM+ cells (approximately 3×105), obtained from biliary tissue specimens, were seeded onto 3 cm diameter plastic culture dishes and kept overnight (˜12 hours) in KM with 10% FBS. Thereafter, cell cultures were maintained in serum-free KM and observed for at least 2 months. For testing the clonal expansion of hBTSCs, a single cell suspension was obtained, and the cells were plated on culture plastic at a clonal seeding density (500/cm2)33 in KM, conditions under which they self-replicate every ˜36-40 hrs indefinitely (especially if at low (2%) oxygen conditions). Hepatoblasts last only about 5-7 days under these conditions (they require additional factors for longer term survival and expansion). Mature epithelial cells of liver, biliary tree and pancreas do not survive beyond a week in serum-free KM.

### Cell Viability.

Cell viability was determined by trypan blue exclusion assay (Sigma #302 643-25G). The cells staining blue were dead; the viable cells did not stain. This dye was used 1:1 v/v with the cell solution. The cell count was carried out through the use of FAST-READ 102 (Biosigma#BSV100). Cells viability was calculated immediately after cell thawing.

### Senescence.

Senescence of thawed cells was determined by the X-Gal test (Sigma #CS0030)34. We used a cell density of 2.6×104 cells/cm2, and the cells were grown for three days before testing. The cells cryopreserved in Sol1 and Sol3, ones that had demonstrated the highest viabilities on thawing, were analysed further with the X-Gal test. The results were compared with controls: cells that had not been cryopreserved. The controls comprised cells that had been cultured, detached and then plated for the assay to imitate the process generating freshly isolated cells.

### Population Doubling.

The proliferation rate was analysed on the same hBTSC population, seeded in 6 multi-well plates at the density of 1×10⁴ cell/cm² and cultured for 7 days. The cell counts were performed under the following culture conditions:
hBTSCs cryopreserved in Sol1
hBTSCs cryopreserved in Sol3
hBTSCs freshly isolated (not cryopreserved)

The medium was changed every three days, using serum-free KM. The mean of the cell number was calculated on three experimental samples for each condition, and cell density was expressed as the mean of cells/cm²±standard deviation (SD). Cells were detached from supports and were counted by trypan blue assay. For these experiments we used only viable cells.

The PDT was calculated in the phase exponential growth by the following equation (1)³⁵:

PDT=log₁₀×ΔT/log₁₀(N_{7d})−log₁₀(N_{1d}) (24)

N_{7d} is the cell number at day 7, and N_{1d} is the cell number at day 1.

To determine the PD rate, hBTSCs, were initially seeded at the density of 1×10⁴ cell/cm² in culture medium. Three samples for each condition were used. The following equation (2)³⁵ was applied:

PD=log₁₀(N)−log₁₀(N)/log₁₀(2) (2)

N is the harvested cell number and Nₛ is the initial plated cell number.

### Colony Counting

The hBTSC colonies began to appear between 1 and 2 weeks after plating and were easily identified by inspection at 10× with a light microscope. Any size colony was counted as one, whether large ones at >3,000 cells or small ones at <200 cells. Each well of the 8 well chamber slide was evaluated using 10× magnification for colonies and counted after 2-3 weeks of culture. Observations of colony number, size, and morphology were noted. Given that the highest viabilities on thawing were given by cells cryopreserved in buffers Sol1 and Sol3, these cells were subjected to further assays to assess their responses to freezing¹⁹.

### Quantitative Reverse-Transcription Polymerase Chain Reaction (RT-qPCR) Analysis.

RNA extractions were performed on tissues from mouse liver or from hBTSC cultures. Total RNA from intrahepatic and extrahepatic biliary tree-derived cell cultures was extracted by the procedures of Chomczynski and SacchiI³⁶. We have used the GAPDH and B3-ACTIN as reference genes for in vitro and in vivo data respectively.

RNA quality and quantity were evaluated with the Experion Automated Electrophoresis System RNA equipped with the RNA StSens Analysis Chip (Bio-Rad Laboratories, Hercules, Calif., USA) as previously described. The expression of the genes was conducted by reverse-transcription and qPCR amplification performed in a closed tube (OneStep RT-qPCR by Qiagen, Hamburg, Germany) on total RNA samples extracted from cells and tissues. These genes were co-amplified with the GAPDH housekeeping gene used as a reference. The gene expression was measured by the quantification of amplicons with on-chip capillary microelectrophoresis performed with the Experion System (Bio-Rad, UK). The expression of the gene of interest was calculated by the ratio of the concentrations of the gene of interest and the reference gene GAPDH in vitro and β-actin in vivo (reported by instrument in nmol/L)

The following genes of interest (GOI) were amplified using the primer pairs reported for each of them. The ratio of concentrations of GOI and the reference genes, namely, GAPDH for CDH1, CD44, ITGB1/4, SOX2/17, PDX1, EpCAM, NANOG, OCT, CYP3A4, TRANSFERRIN, SR, ASBT, CFTR, INS, GLUCAGON, and beta-actin for human and murine albumin, was assumed to be the GOI relative expression.

| Gene | Id. Sequence | Primers (5′-3′) |
|---|---|---|
| CDH 1 E-Cadherin | NM_004360.3 | TCACAGTCACTGACACCAACGA |
| | | GGCACCTGACCCTTGTACGT |
| CD 44 Hyaluronic acid receptor | NM_000610.3 | TGCCGCTTTGCAGGTGTAT |
| | | GGCCTCCGTCCGAGAGA |
| ITGB 1 Integrin β 1 | NM_002211.3 | CAAAGGAACAGCAGAGAAGC |
| | | ATTGAGTAAGACAGGTCCATAAGG |
| ITGB 4 Integrin β 4 | NM_000213.3 | CTGTGTGCACGAGGGACATT |
| | | AAGGCTGACTCGGTGGAGAA |
| GAPDH | NM_002046.3 | AAGGTGAAGGTCGGAGTCAA |
| | | AATGAAGGGGTCATTGATGG |
| Human albumin | NM_000477.5 | AGAGGTCTCAAGAAACCTAGGAAA |
| | | GGTTCAGGACCACGGATAGA |
| Mus musculus albumin | NM_009654.3 | CGAGAAGCTTGGAGAATATGGA |
| | | CTTGGTGCCCACTCTTCCTA |
| Mus musculus beta-actin | NM_007393.5 | GGATGCAGAAGGAGATTACTGC |
| | | CCACCGATCCACACAGAGTA |
| SOX2 | NM_003106 | TCGAGAACCGAGTGAGAGG |
| | | GCAAAGCTCCTACCGTACCA |
| SOX17 | NM_022454 | AAGATGCTGGGCAAGTCGTGG |
| | | CTTGTAGTTGGGGTGGTCCTG |
| PDX1 | NM_000209 | CATTGGAAGGCTCCCTAACA |
| | | TTCCACTGGCATCAATTTCA |
| EpCAM | NM_002354.2 | CCATGTGCTGGTGTGTGA |
| | | TGTGTTTTAGTTCAATGATGATCCA |
| Nanog | NM_000615 | AGATGCCTCACACGGAGACT |
| | | GGTCCTCTCCTCCTCCGTTCG |
| OCT4 | NM_002701 | TCGAGAACCGAGTGAGAGG |
| | | GAACCACACTCGGACCACA |
| CYP3A4 | NM_017460 | AAGTCGCCTCGAAGATACACA |
| | | AAGGAGAGAACACTGCTCGTG |
| TRANSFERRIN | NM_001063 | CCTCCTACCTTGATTGCATCAG |
| | | TTTTGACCCATAGAACTCTGCC |
| SR | NM_002980.2 | CTCAATGGGGAGGTGCAGCTGGA |
| | | CTCTCAGATGATGCTGGTCCTG |
| ASBT | NM_000452 | TGTGTTGGCTTCCTCTGTCAG |
| | | GGCAGCATCCTATAATGAGCAC |
| CFTR | NM_000492 | AAAAGGCCAGCGTTGTCTCC |
| | | TGAAGCCAGCTCTCTATCCCA |
| INS | NM_000207; | GCAGCCTTTGTGGAACCAACAC |
| | NM_001180597 | CCCCGCACACTAGGTAGAGA |
| GLUCAGON | NM_002054 | GACAAGCGCCATTCACAGG |
| | | TGACGTTTGGCAATGTTATTCCT |

Measurement of Albumin Secretion in hBTSCs

The hBTSCs underwent a self-replication period in serum-free Kubota's Medium (KM) after plating on culture plastic. Cells were seeded at the density of 3.8×105 cells/cm2 in KM. The medium was changed every 3 days. After 1 week of culturing in KM, the cultures were subjected either to KM (controls) or to an HDM tailored for hepatocytes. The albumin secretion experiment was performed after a further 2 weeks of culturing. For the entire period of the assay, the cells were not passaged.

Cell culture medium collected over 24 hours was analysed in triplicate by the human albumin-specific ELISA kit (Albumin Human ELISA Kit, Abcam, Cambridge, UK, catalog# ab108788). Medium was collected and stored at −20° C. Values are expressed as micrograms per million cells per 30 milliliter culture medium. The evaluation of the human albumin secretion in the supernatant medium has been also performed in HepG2 cells purchased commercially from Lonza (Basel, Switzerland), a well differentiated human hepatocellular carcinoma cell line, utilized as a positive control.

Measurement of C-Peptide Secretion in hBTSCs

The hBTSCs underwent a self-replication period in serum-free KM after plating on culture plastic. Cells were seeded at the density of 5.2×10⁵ cell/cm² in KM. The medium was changed every 3 days. After 1 week of culturing in KM, the cultures were subjected either to KM (controls) or to an HDM tailored for differentiation of the stem cells to pancreatic islets. The glucose challenge experiment was performed after further 2 weeks of culturing. For the entire period of the assay, the cells were not passaged.

Cells were washed three times with Dulbecco's Phosphate Buffered Saline (DPBS, GIBCO, Catalog#14190144). Afterwards cells were incubated for 2 hours with Connaught Medical Research Laboratories medium (CMRL) with 5.5 mM glucose and antibiotics; CMRL is a chemically defined, protein-free medium with higher levels of nucleosides and vitamins and found useful for human and primate cells. The incubation medium was collected and stored at −20° C. Cells were again gently washed three times with DBPS and then incubated for 2 hours in glucose-free CMRL supplemented with 28 mM glucose and antibiotics. Again, medium from each well was collected and stored at −20° C. Cells were counted using Trypan Blue assays. Samples from cultures at 5.5 mM versus 28 mM glucose were used for assays of C-peptide synthesis. The human C-peptide content in the medium was measured by an ELISA kit (R&D, Ref DICP00) and normalized to the cell number of each sample. The amount of C-peptide generated in response to the high-glucose challenge was divided by the amount generated by the low-glucose challenge to yield the mean C-peptide secretion index. The stimulation index of C-peptide secretion is calculated as the ratio between C-peptide secreted in the medium under high glucose concentration and C-peptide secreted under basal (low) glucose concentration; C-peptide concentration in the medium was quantified by ELISA in the same cell sample and during a fixed time period (2 h).

### Cell Transplantation in SCID Mice.

All animal experiments have been carried out in accordance with the EU Directive 2010/63/EU for animal experiments and with Sapienza institutional guidelines. The animal experimental protocol was reviewed and approved by the Ethic Committee of Sapienza University of Rome and Umberto I University Hospital of Rome (Prot. 541). SCID mice (T/SOPF NOD.CB17 PRKDC/J) (N=4) were male, 4-week old animals and were used as the hosts for transplantation of human cells. Animals were sedated with an anaesthetic drug (2, 2, 2-Tribromoethanol). Thereafter, freshly isolated or cryopreserved and thawed 2×10⁶ hBTSCs were suspended in 100 μl saline and injected into the liver via the spleen. Sham controls were infused only with 100 μl saline. All the animals were closely monitored until recovery, and were allowed free access to food and water. All the animal protocols complied with our institutional guidelines. No mortality occurred. At 30 days after cell transplantation, mice were sacrificed, and the livers removed for further analyses. Liver samples were placed in Trizol Reagent for gene analyses or in 4% formalin for pathologic and immunohistochemistry analyses. Blood samples were collected from the heart, centrifuged and serum samples stored at −20° C. for quantification of human albumin by ELISA (ABCAM #ad108788).

### Light Microscopy (LM), Immunohistochemistry (IHC) and Immunofluorescence (IF)

Specimens were fixed in 10% buffered formalin for 2-4 hours, embedded in low-temperature-fusion paraffin (55-57° C.), and 3-4 m sections were stained with haematoxylin-eosin and Sirius red/Fast green, according to standard protocols. For IHC, endogenous peroxidase activity was blocked by 30 min incubation in methanolic hydrogen peroxide (2.5%). Antigens were retrieved, as indicated by the vendor, by applying Proteinase K (Dako, code Sol3020) for 10 min at room temperature. Sections were then incubated overnight at 4° C. with primary antibodies.

### Primary Antibodies

| Name | Host/isotype | Source | Catalog# | Dilution | Application |
|---|---|---|---|---|---|
| Albumin | Rabbit IgG | Abcam | AB108788 | 1:100 | ELISA |
| SOX9 | Rabbit IgG | Millipore | AB5809 | 1:100 | IHC |
| Insulin | Guinea | DAKO | IS002 | 1:100 | IHC and |
| | Pig IgG | | | | ELISA |
| CK19 | Mouse IgG1 | DAKO | MO888 | 1:100 | IHC/IF |
| Anti- | Mouse IgG1 | Chemicon | MAB1273 | 1:200 | IHC |
| Human | | | | | |
| Mito- | | | | | |
| chondria | | | | | |
| SOX17 | Goat IgG | R&D | AF1924 | 1:50 | IHC/IF |

Samples were rinsed twice with PBS for 5 min, incubated for 20 min at room temperature with secondary biotinylated antibody (LSAB+ System-HRP, Dako, code K0690; Glostrup, Denmark) and then with Streptavidin-HRP (LSAB+ System-HRP, Dako, code K0690). Diaminobenzidine (Dako) was used as substrate, and sections were counterstained with haematoxylin. For immunofluorescence on cell culture, slides chambers were fixed in acetone for 10 min at room temperature and then rinsed with PBS-Tween 20. Non-specific protein binding was blocked by 5% normal goat serum. Fixed cells were incubated with primary antibodies. Then, cells were washed and incubated for 1 h with labelled isotype-specific secondary antibodies (anti-mouse AlexaFluor-546, anti-mouse Alexafluor-488, anti-rabbit Alexafluor-488, anti-goat AlexaFluor-546, Invitrogen, Life Technologies Ltd, Paisley, UK) and counterstained with 4,6-diamidino-2-phenylindole (DAPI) for visualization of cell nuclei. For all immunoreactions, negative controls (the primary antibody was replaced with pre-immune serum) were also included. Sections/Cultures were examined in a coded fashion by Leica Microsystems DM 4500 B Light and Fluorescence Microscopy (Weltzlar, Germany) equipped with a JenoptikProg Res C10 Plus Videocam (Jena, Germany). IF staining was also analysed by Confocal Microscopy (Leica TCS-SP2). LM, IHC and IF observations were processed with an Image Analysis System (IAS—Delta Sistemi, Roma—Italy) and were independently performed by two pathologists in a blind fashion.

### Positive and Negative Controls

| | | | NEGATIVE |
|---|---|---|---|
| ANTIGEN | METHODS | POSITIVE CONTROL | CONTROL |
| Albumin | ELISA | Mature human | hBTSCs |
| | | hepatocytes | in KM |
| human | IHC/IF | Human liver | Mouse Liver |
| mithocondria | | | |
| Hep-Par1 | IHC/IF | Human liver | Mouse Liver |
| human Albumin | IHC/IF | Human liver | Mouse Liver |

All counts have been performed in six non-overlapping fields (magnification ×20) for each slide; at least 3 different slides have been taken from each specimen. For IHC/IF staining, the number of positive cells was counted in a random, blinded fashion in six non-overlapping fields (magnification ×20) for each slide/culture, and the data are expressed as % positive cells.

### Statistical Analysis

Data were expressed as mean±SD. Statistical analyses were performed by SPSS statistical software (SPSS Inc. Chicago Ill., USA). Differences between groups for non-normal distribution parameters were tested by Mann-Whitney U tests. Statistical significance was set to a p-value <0.05.

II. Results

Viability, Senescence and Colony Formation by Cryopreserved hBTSCs.

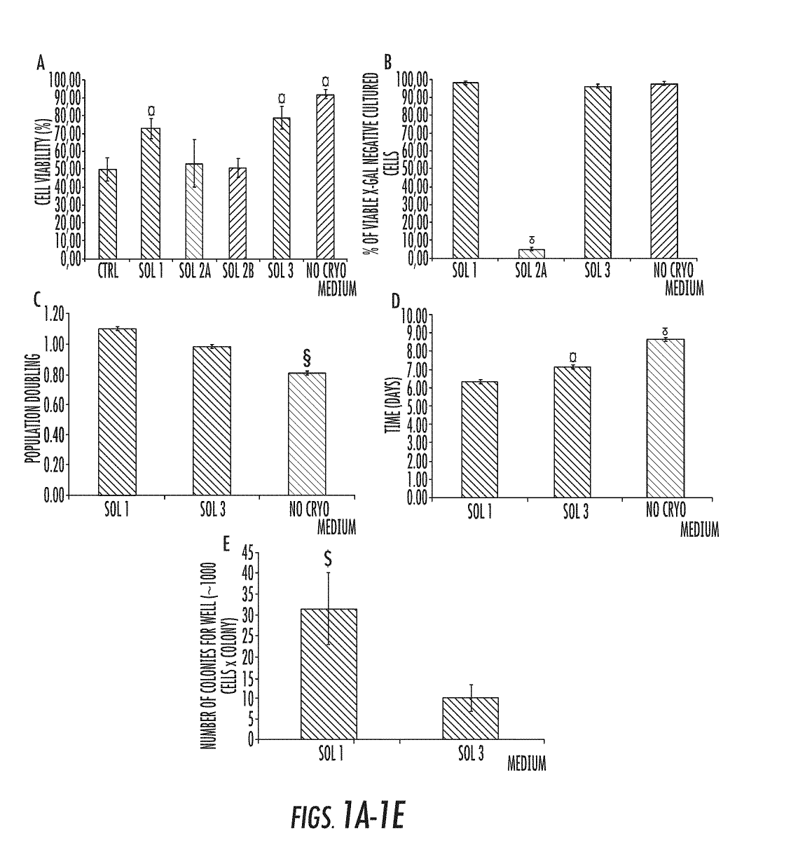

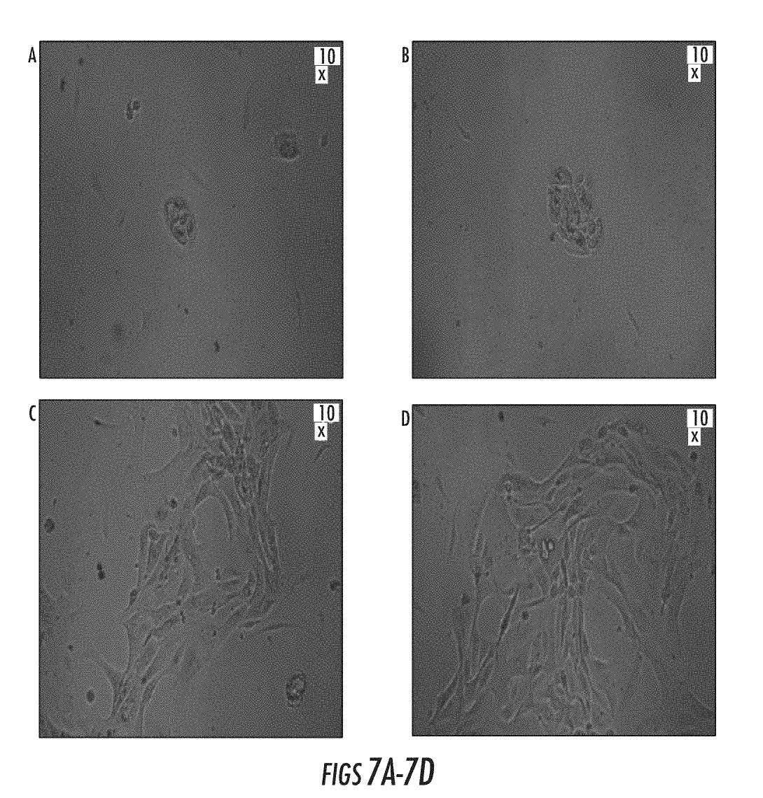

The hBTSCs were cryopreserved in a basal control solution (10% DMSO, 1.5% human albumin in KM) for 4-12 weeks and, then were thawed and seeded at a density of 10,000 cells/mL on plastic. FIGS. 1 and 7 show the cell viability and morphology of hBTSC cultures after 4 weeks of cryopreservation in the basal control solution. After thawing, cells were grown for a period of 30 days in Kubota's Medium (KM). The hBTSCs were able to form cell colonies that were morphologically similar to those generated by freshly isolated cells (FIG. 7). We tested various cryopreservation buffers. All of them were comprised of serum-free KM supplemented with 10% dimethyl-sulfoxide (DMSO) and with distinctions in containing different concentrations of human albumin and HA. The percent of viable cells was assessed after 4 weeks of cryopreservation and immediately after thawing (N=9). Cells in Solution 1 (Sol1: supplemented further with 0.1% HA+15% recombinant human albumin) had an average viability of 72.78±5.65%. Those in Solution 3 (Sol3: supplemented further with 15% recombinant human albumin) had an average viability of 78.89±6.51%. Sol1 and Sol3 yielded viabilities after thawing that were significantly higher (p<0.001) than those in the other buffers. The average viabilities in Solution 2A (Sol2A: supplemented with 0.1% HA) were 53.33±13.23%; those in Solution 2B (Sol2B: supplemented with 0.05% HA) were 50.56±5.27%, and those in the control solution (CTRL: supplemented with 1.5% recombinant human albumin) were 50.00±⁶0.61%. No significant difference in cell viability was found between Sol1 and Sol3 (FIG. 1A). Applicants next evaluated cell senescence (X-Gal) in cultures obtained from cryopreserved or freshly isolated cells from the same donors. The number of X-Gal negative cells (not senescent) exceeded 95% after cryopreservation (FIG. 1B). No difference was observed between Sol1 (with HA; 98.57±0.36; N=9) and Sol3 (without HA; 96.72±0.66; N=9; p>0.05), and between cryopreserved and freshly isolated cells (98.00±0.53; N=9; p>0.05). Senescent negative cells were markedly lower in Sol2A (4.85±0.80; N=9; p<0.0001) than other conditions. Cell population doubling (PD) in cultures confirmed the optimal maintenance of the in vitro functional properties of the hBTSCs cryopreserved in Sol1 and Sol3. The PD in fact, was significantly higher in Sol1 (1.11±0.01) and Sol3 (0.98±0.01) as compared to those that were freshly isolated (0.81±0.01) (N=8; p<0.01) (FIG. 1C). The PD time (PDT) was significantly lower in Sol1 (with HA) than Sol3 (without HA) (6.32±0.02 vs 7.14±0.02 days; N=8; p<0.001), and in Sol3 as compared to freshly isolated cells (8.67±0.03 days) (N=8; p<0.0001) (FIG. 1D).

Colony formation is a surrogate marker of seeding and engraftment capacity. The number of colonies, formed by 200-3,000 cells, was dramatically increased in cells cryopreserved in Sol1 (with HA, 31.56±8.43, N=18) as compared to those in Sol3 (without HA, 10.11±3.85, N=18; p<0.000001) (FIG. 1E).

Expression of Stem Cell Markers and Adhesion Molecules in Cryopreserved hBTSCs.

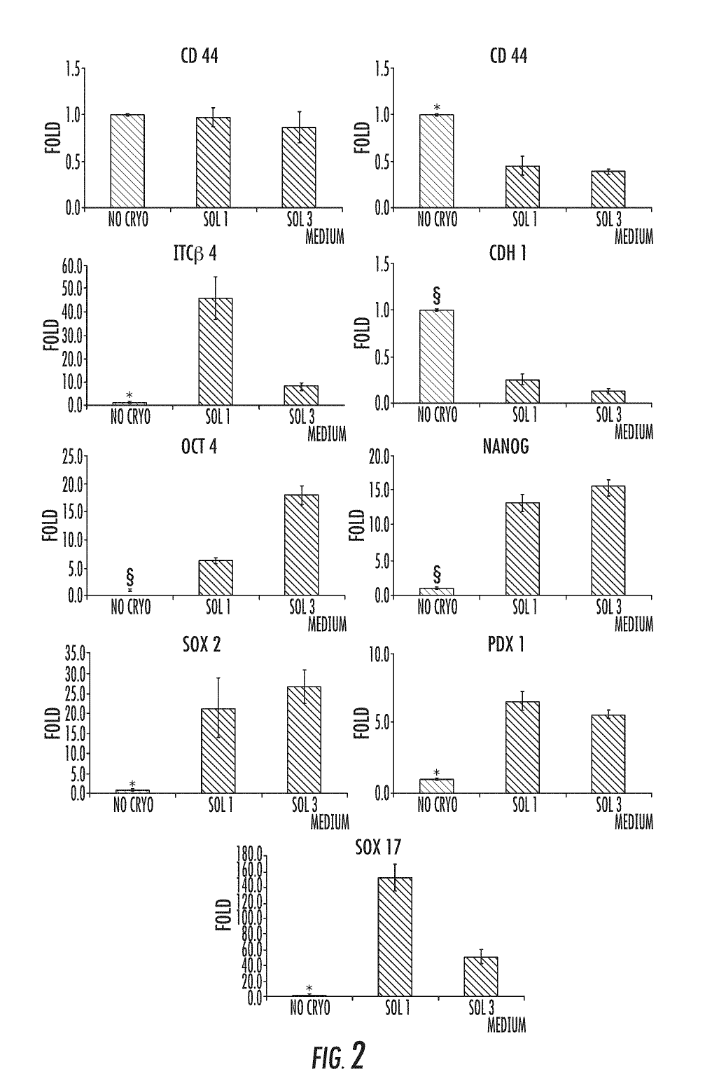

To evaluate whether cryopreservation affects stem cell phenotype, the expression of pivotal genes commonly expressed by endodermal stem cells was assessed. These include pluripotency genes (OCT4, NANOG, SOX2) and endodermal transcription factors (SOX17, PDX1). These were assessed before and after 1 month of cryopreservation. Interestingly, stem cell genes were more highly expressed in hBTSCs cryopreserved in Sol1 and Sol3 than in freshly isolated cells [SOX2 (p<0.05), PDX1 (p<0.05), NANOG (p<0.01), SOX17 (p<0.05), and OCT4 (p<0.01); N=5] (FIG. 2).

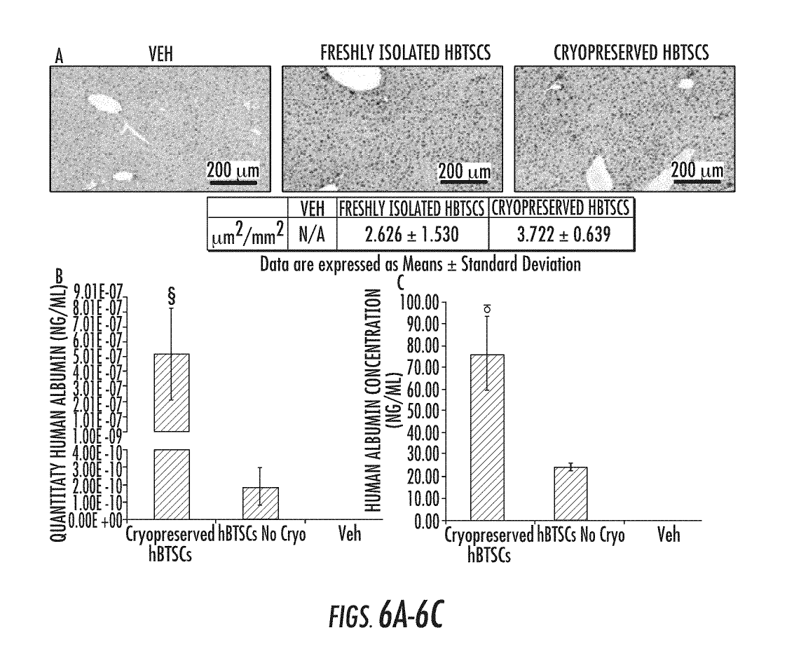

As shown by Turner et al.²⁰ engraftment after cell transplantation well correlates with the level of expression of adhesion molecules. Therefore, Applicants analysed by RT-qPCR the gene expression of different genes encoding adhesion molecules including CD44 (the hyaluronan receptor), ITGB1 (integrin beta1), ITGB4 (integrin beta 4), and CDH1 (cadherin 1). No significant differences were found in cells subjected to different cryopreservation buffers versus freshly isolated cells in the expression of CD44 (FIG. 2), while the expression of ITGB1 and CDH1 was decreased in cryopreserved cells compared to freshly isolated hBTSCs (ITGB1, p<0.05; CDH1N=5; p<0.01) (FIGS. 6B and 6C); ITGB4 expression increased in cryopreserved hBTSCs (p<0.05) (FIG. 2).

Multipotency is Preserved with Cryopreservation.

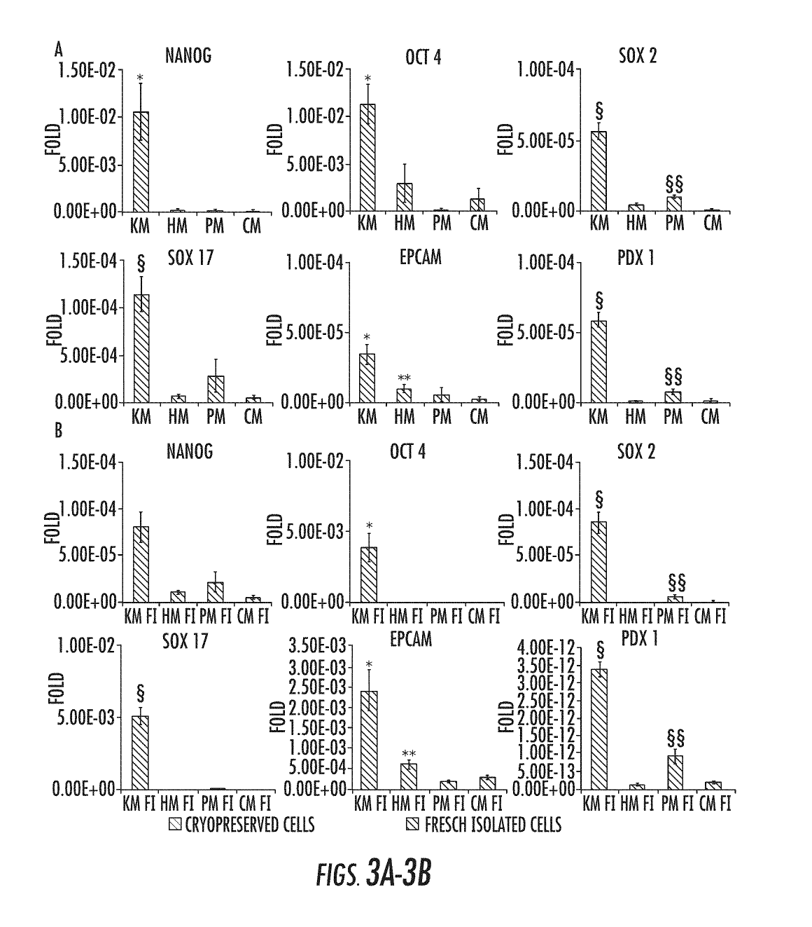

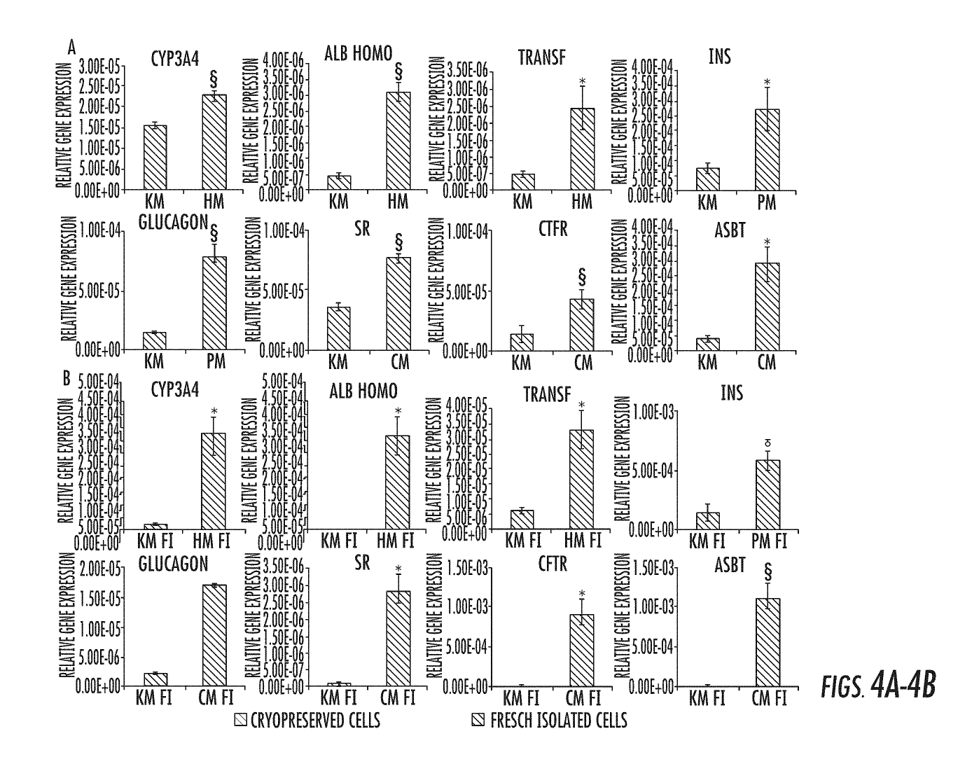

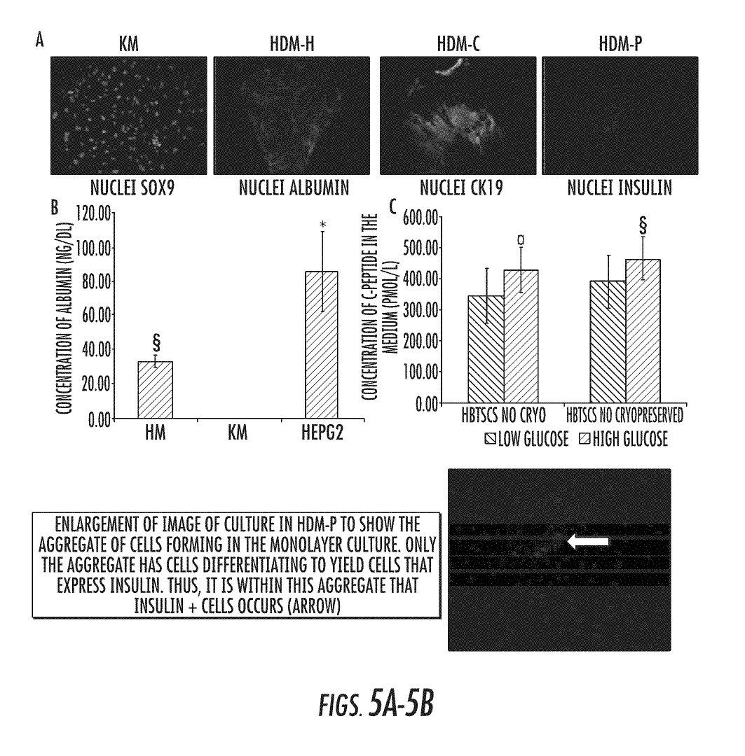

Multipotency genes are expressed in hBTSCs under self-renewal conditions and then disappear upon differentiation towards mature cells. Applicants tested cultures of hBTSCs after cryopreservation in Sol1 (FIG. 3A, 4A), Sol3 (data not shown) versus freshly isolated cells (FIG. 3B, 4B). For differentiation conditions, Applicants used different hormonally defined media (HDM) specifically tailored to induce the differentiation of hBTSCs to mature hepatocytes (HM), cholangiocytes (CM) or pancreatic islets (PM). KM without hydrocortisone was used as a control since this medium is permissive for cell expansion and is neutral for differentiation towards both liver and for pancreas (glucocorticoids must be avoided for pancreatic differentiation). Cryopreserved hBTSCs (FIG. 3A), as well as freshly isolated cells (FIG. 3B), showed decreased expression of the pluripotency genes (NANOG, OCT4, and SOX2) and endodermal stem cell genes (EpCAM, PDX1, and SOX17) after two weeks in culture in HDMs tailored for differentiation of the stem cells to hepatocytic (HM), pancreatic (PM) or biliary (CM) fates (p<0.05). When hBTSCs (Sol1 and freshly isolated) were transferred from KM to HM for 2 weeks, significant increases in expression of mature hepatocyte-specific genes were observed including (e.g. Albumin (Alb); N=5; p<0.01 vs KM; Transferrin (Transf); N=5; p<0.05 vs KM and Cytochrome P450 3A4 (CYP3A4); N=5; p<0.01 vs KM) (FIG. 4). Similarly, when hBTSCs (Sol1 and freshly isolated) were transferred into PM or CM for 2 weeks, significant increases of pancreatic islet-specific gene expressions (Insulin (Ins), N=5, p<0.05; Glucagon N=5, p<0.01 PM vs KM), and of large cholangiocytes-specific gene expressions (Secretin Receptor (SR), N=5, p<0.01; Cystic fibrosis transmembrane conductance regulator (CFTR), N=5, p<0.01; Apical sodium dependent bile acid transporter (ASBT), N=5, p<0.05 CM vs KM) (FIG. 4) were observed. The hBTSCs in HDMs developed characteristic changes in morphology and phenotypic traits. Specifically, after 15 days in HM, cuboidal-shaped cells expressing albumin (hepatocyte markers) were observed (FIG. 5A) (N=5); after 15 days in CM, clusters of cells expressing Cytokeratin 19 (CK19) appeared (FIG. 5A) (N=5); while, hBTSCs in PM produced, after 14 days, dense balls of aggregated cells budding from the edges of the colonies and containing cells expressing insulin (FIG. 5A) (N=5). No significant differences were observed between Sol1, Sol3 and freshly isolated cells (N=5).

Applicants then evaluated at a functional level how cryopreserved hBTSCs can be effectively differentiated in vitro into hepatocyte-like cells or pancreatic islet-like cells. Cryopreserved hBTSCs cultured in HM acquired the capacity to produce and secrete albumin (N=7; p<0.01 in HM vs KM) although at a slight lower extent with respect to HepG2 (p<0.05) (FIG. 5B). When cultured in PM, hBTSCs acquired insulin secretion that was regulated by glucose concentration (low versus high glucose concentration; N=7; p<0.01 vs low glucose) (FIG. 5C).

Effective In Vivo Engraftment of Cryopreserved hBTSCs.

To determine whether cryopreserved hBTSCs can effectively engraft and proliferate in the livers of immune-compromised mice, cells were transplanted into the spleen of SCID mice. The livers were then analysed 30 days after cell transplantation by immunohistochemistry with an antibody to human mitochondria as previously described 5. As shown in FIG. 6A, cryopreserved (Sol1) hBTSCs engrafted into liver parenchyma with the same efficiency as freshly isolated cells (N=3; p>0.05). Indeed, the expression of human mitochondria in liver parenchyma of the SCID mice indicated that 2.626±1.530% and 3.722±0.639 of the host parenchymal cell mass comprised human cells derived from freshly or cryopreserved hBTSCs, respectively (FIG. 6A). To confirm the effective engraftment and differentiation of transplanted cryopreserved hBTSCs into the murine livers, we measured human albumin mRNA in the liver and human albumin (protein) in the serum. The expression of human albumin mRNA in the liver was significantly higher (N=3; p<0.01) in mice transplanted with cryopreserved hBTSCs (5.19*10−7±3.06*10−7) than mice transplanted with freshly isolated hBTSCs (1.90*10−10±1.09*10−10) (FIG. 6B). Accordingly, in the same animals, the human serum albumin levels were significantly higher (N=3; p<0.0001) in mice transplanted with cryopreserved hBTSCs (76.39±17.04 ng/mL) than mice transplanted with freshly isolated hBTSCs (24.13±1.44 ng/mL) (FIG. 6C).

III. Discussion

Applicants have established a successful cryopreservation protocol for hBTSCs comprised of serum-free Kubota's Medium (KM) supplemented with DMSO (10%), HA (0.1%) and high concentrations of recombinant human albumin (15%). The key findings leading to this conclusion are that: 1) hBTSCs can survive and have a high viability on thawing (˜80%) after 120 days of cryopreservation if subjected to this cryopreservation buffer; 2) the in vitro proliferation rate (population doubling times) and colony formation capacity were improved by supplementation of cryopreservation buffers with HA (0.1%); 3) hBTSCs cryopreserved in buffers containing high albumin concentrations+HA, efficiently differentiated in vitro to mature fates (hepatocytes, cholangiocytes, or functional pancreatic β-cells); 4) hBTSCs cryopreserved in the buffer containing high albumin concentrations+HA effectively engrafted and differentiated in vivo after transplantation in SCID mice.

Applicants note that they did cell isolation and cryopreservation/thawing processes under GMP conditions; only in vitro experiments were not GMP-grade strategies of cell cryopreservation. Applicants aim to protect mechanisms of cell viability and proliferative capacity and are based on the use of isotonic buffers, antifreeze proteins (from arctic animals), antioxidants and freezing reagents such as DMSO or glycerol. The existing methods work well for hematopoietic cell subpopulations, since they inherently have extracellular matrix components that are missing cell binding domains and so the cells are able to float. Thus, their adhesion and other matrix-dependent functions are intact and not adversely affected by cryopreservation. By contrast, isolation of cells from solid organs requires enzymatic activity that dissolves the matrix enabling the cells to be dispersed into cell suspensions, making these cells vulnerable to adverse effects of cryopreservation on matrix-dependent activities²⁰. Liver cells, including hepatocytes, are representative of cells from solid organs and demonstrating enormous difficulties encountered in cryopreservation¹⁴. In addition, cryopreservation of stem/progenitor cells has additional obstacles over those for mature cells, since many additives of cryopreservation buffers, such as serum, can eliminate stemness traits and, in parallel, trigger differentiation²⁰. Applicants demonstrated previously that these obstacles can be overcome by using an isotonic medium such as Cryostor (Crystor-10) or a wholly defined, serum-free stem cell medium, KM supplemented with hyaluronans, a dominant constituent of the matrix chemistry of stem cell niches²¹. In this study we were able to improve the conditions further by adding high levels of recombinant human albumin (final concentration: ˜3%). Applicants evaluated the maintenance, after cryopreservation, of key cell phenotypic properties such as viability, seeding, proliferation rate and differentiation potential^{1, 19}. Firstly, Applicants observed that cryopreservation in either serum-free buffer (Sol1 or Sol3) containing high levels of recombinant human albumin (˜3%) resulted in a significantly better cell viability, after thawing, when compared to cells in the other buffers tested. Therefore, supplementation with high concentrations of human albumin (˜3% compared to 0.3%) facilitates the maintenance of cell viability after thawing. Previously, Terry et al.¹⁴ had proposed that human serum albumin could represent an alternative to foetal serum assuming that the high levels of albumin contained in the serum is the major determinant of the serum cryo-protective effect; our results confirmed the role of albumin as a cryo-protective agent.

Applicants further demonstrated that cryopreservation in solutions containing high albumin concentration±HA protects hBTSCs from cell senescence. Cell senescence is correlated with telomere shortening during cell divisions, but, stem cells counteract senescence through high telomerase activity^{22, 23}, and this has been demonstrated by Reid and associates in hepatic stem cells^{22, 23}. Proliferation rates in vitro have been analysed by population doubling assays in which we demonstrated the preservation of the proliferation capabilities by cryopreserved hBTSCs with respect to freshly isolated cells. Seeding and proliferation are both correlated with colony formation capacity²⁰. Applicants tested whether the colony formation properties are influenced by any of the cryopreservation buffers. Expression of some adhesion molecules (e.g. ITGB4) was improved and that of CD44 unaffected, whereas others were reduced (ITGB1, CDH1). Still proliferation in cells cryopreserved in Sol1 versus Sol3 were similar, but with dramatic increases in colony formation in those in Sol1 containing both the high albumin levels and also HA. It is noteworthy that all subpopulations of stem cells and progenitors in the liver express CD44, the receptor for HA, and that apoptosis is increased in cells that are unable to regain adhesion proteins quickly following thawing²⁰. HA represents the main constituent of the liver stem cell niche²⁴. Turner et al.²⁰ observed that the use of supplementation with hyaluronans constitutes a successful option for an optimal cryopreservation of human hepatic stem/progenitor cells (hHpSC). Here Applicants demonstrated a positive role of HA as a preconditioning agent which could favour the engraftment of cells after cryopreservation. Indeed, data obtained by RT-qPCR demonstrated that in hBTSCs cryopreserved in Sol1, the expression of adhesion molecules is partially preserved, while genes of pluripotency and endodermal stem cells are entirely preserved, as compared to expression in freshly isolated cells. These data are in agreement with the previous results by Turner et al.²⁰. Finally and most importantly, the differentiation potential of hBTSCs was unaffected and similar to that of freshly isolated cells when cryopreserved in Sol1 or 3 containing high albumin concentration±HA^{1-4, 25}. Indeed, Applicants showed in vitro, in media specifically tailored to induce the selective differentiation of hBTSCs to hepatocytes, cholangiocytes or pancreatic cells, that the differentiation capacities are also well preserved by our protocol of cryopreservation. They are not influenced by HA. This has also been demonstrated at a functional level by evaluating the albumin synthesis/secretion capacity of cells differentiated toward hepatocytes and insulin production, in both basal conditions and after glucose challenge, in cells differentiated toward pancreatic cells.

Finally and most importantly, Applicants demonstrated that hBTSCs cryopreserved in buffers containing high albumin+HA (Sol1) and transplanted into SCID mice, displayed an engraftment and differentiation efficiency even better than freshly isolated cells. The percent of human cells hosting murine liver and the synthesis and secretion of human albumin were in fact better for cryopreserved than freshly isolated hBTSCs (Sol1 vs freshly isolated). This surprising result is in keeping with observations in vitro in which HA improves cell engraftment and with observations in vivo in which cells coated with HA showed higher rates of liver engraftment, after transplantation, than freshly isolated cells²⁶.

The hBTSCs are easily isolated under GMP conditions from human tissues of donors of any age and have already been used for cell therapy of patients with advanced liver cirrhosis²⁷. Given the extremely wide availability of biliary tree tissues for their isolation and given their biological characteristics, hBTSCs have enormous applicative potential for regenerative medicine of the liver and pancreas, including diabetes. In this study, hBTSCs were successfully cryopreserved without loss of crucial cell functions; this facilitates the establishment of a cell bank of hBTSCs that can be stored and used rapidly offering logistical advantages for cell therapies of liver diseases.

### REFERENCES

1. Cardinale, V. et al. Multipotent stem/progenitor cells in human biliary tree give rise to hepatocytes, cholangiocytes, and pancreatic islets. Hepatology 54, 2159-2172 (2011).
2. Cardinale, V. et al. The biliary tree—a reservoir of multipotent stem cells. Nature reviews. Gastroenterology & hepatology 9, 231-240 (2012).
3. Carpino, G. et al. Biliary tree stem/progenitor cells in glands of extrahepatic and intraheptic bile ducts: an anatomical in situ study yielding evidence of maturational lineages. Journal of anatomy 220, 186-199 (2012).
4. Semeraro, R. et al. The fetal liver as cell source for the regenerative medicine of liver and pancreas. Annals of translational medicine 1, 13 (2013).
5. Carpino, G. et al. Evidence for multipotent endodermal stem/progenitor cell populations in human gallbladder. Journal of hepatology 60, 1194-1202 (2014).
6. Riccio, M. et al. The Fas/Fas ligand apoptosis pathway underlies immunomodulatory properties of human biliary tree stem/progenitor cells. Journal of hepatology 61, 1097-1105 (2014).
7. Lanzoni, G. et al. Concise review: clinical programs of stem cell therapies for liver and pancreas. Stem cells 31, 2047-2060 (2013).
8. Cardinale, V. et al. Transplantation of human fetal biliary tree stem/progenitor cells into two patients with advanced liver cirrhosis. BMC gastroenterology 14, 204 (2014).
9. Novicki, D. L., Irons, G. P., Strom, S. C., Jirtle, R. & Michalopoulos, G. Cryopreservation of isolated rat hepatocytes. In vitro 18, 393-399 (1982).
10. Lozoya, O. A. et al. Regulation of hepatic stem/progenitor phenotype by microenvironment stiffness in hydrogel models of the human liver stem cell niche. Biomaterials 32, 7389-7402 (2011).
11. Dixit, V., Darvasi, R., Arthur, M., Lewin, K. & Gitnick, G. Cryopreserved microencapsulated hepatocytes—transplantation studies in Gunn rats. Transplantation 55, 616-622 (1993).
12. Guyomard, C., Rialland, L., Fremond, B., Chesne, C. & Guillouzo, A. Influence of alginate gel entrapment and cryopreservation on survival and xenobiotic metabolism capacity of rat hepatocytes. Toxicology and applied pharmacology 141, 349-356 (1996).
13. Canaple, L. et al. Maintenance of primary murine hepatocyte functions in multicomponent polymer capsules—in vitro cryopreservation studies. Journal of hepatology 34, 11-18 (2001).
14. Terry, C., Dhawan, A., Mitry, R. R. & Hughes, R. D. Cryopreservation of isolated human hepatocytes for transplantation: State of the art. Cryobiology 53, 149-159 (2006).
15. Gomez-Lechon, M. J., Lopez, P. & Castell, J. V. Biochemical functionality and recovery of hepatocytes after deep freezing storage. In vitro 20, 826-832 (1984).
16. Rijntjes, P. J., Moshage, H. J., Van Gemert, P. J., De Waal, R. & Yap, S. H. Cryopreservation of adult human hepatocytes. The influence of deep freezing storage on the viability, cell seeding, survival, fine structures and albumin synthesis in primary cultures. Journal of hepatology 3, 7-18 (1986).
17. Alexandre, E. et al. Cryopreservation of adult human hepatocytes obtained from resected liver biopsies. Cryobiology 44, 103-113 (2002).
18. Adams, R. M. et al. Effective cryopreservation and long-term storage of primary human hepatocytes with recovery of viability, differentiation, and replicative potential. Cell transplantation 4, 579-586 (1995).
19. Turner, R., Gerber, D. & Reid, L. The future of cell transplant therapies: a need for tissue grafting. Transplantation 90, 807-810 (2010).
20. Turner, R. A., Mendel, G., Wauthier, E., Barbier, C. & Reid, L. M. Hyaluronan-supplemented buffers preserve adhesion mechanisms facilitating cryopreservation of human hepatic stem/progenitor cells. Cell transplantation 21, 2257-2266 (2012).
21. Wang, Z., Hisatake, G. & Yang, L. Liver-specific deceased donor risk indices. Hepatology research: the official journal of the Japan Society of Hepatology 44, 159-164 (2014).
22. Schmelzer, E. & Reid, L. M. Human telomerase activity, telomerase and telomeric template expression in hepatic stem cells and in livers from fetal and postnatal donors. European journal of gastroenterology & hepatology 21, 1191-1198 (2009).
23. Cardinale, V. et al. Multipotent stem cells in the biliary tree. Italian journal of anatomy and embryology=Archivio italiano di anatomia ed embriologia 115, 85-90 (2010).
24. Wang, Y. et al. Paracrine signals from mesenchymal cell populations govern the expansion and differentiation of human hepatic stem cells to adult liver fates. Hepatology 52, 1443-1454 (2010).
25. Wang, Y. et al. Biliary tree stem cells, precursors to pancreatic committed progenitors: evidence for possible life-long pancreatic organogenesis. Stem cells 31, 1966-1979 (2013).
26. Nevi, L. et al. Hyaluronan coating improves liver engraftment of transplanted human biliary tree stem/progenitor cells. Stem cell research & therapy 8, 68 (2017).
27. Cardinale, V. et al. Adult Human Biliary Tree Stem Cells Differentiate to beta-Pancreatic Islet Cells by Treatment with a Recombinant Human Pdx1 Peptide. PloS one 10, e0134677 (2015).
28. Wauthier, E. et al. Hepatic stem cells and hepatoblasts: identification, isolation, and ex vivo maintenance. Methods in cell biology 86, 137-225 (2008).
29. Schmelzer, E. et al. Human hepatic stem cells from fetal and postnatal donors. The Journal of experimental medicine 204, 1973-1987 (2007).
30. Semeraro, R. et al. Multipotent stem/progenitor cells in the human foetal biliary tree. Journal of hepatology 57, 987-994 (2012).
31. Kubota, H. & Reid, L. M. Clonogenic hepatoblasts, common precursors for hepatocytic and biliary lineages, are lacking classical major histocompatibility complex class I antigen. Proceedings of the National Academy of Sciences of the United States of America 97, 12132-12137 (2000).
32. Harrill, J. A. et al. Lineage-dependent effects of aryl hydrocarbon receptor agonists contribute to liver tumorigenesis. Hepatology 61, 548-560 (2015).
33. Kajstura, J. et al. Evidence for human lung stem cells. The New England journal of medicine 364, 1795-1806 (2011).
34. Fedorovich, N. E. et al. The effect of photopolymerization on stem cells embedded in hydrogels. Biomaterials 30, 344-353 (2009).
35. Pisciotta, A. et al. Human serum promotes osteogenic differentiation of human dental pulp stem cells in vitro and in vivo. PloS one 7, e50542 (2012).
36. Ribaudo, R., Gilman, M., Kingston, R. E., Chomczynski, P. & Sacchi, N. Preparation of RNA from tissues and cells. Current protocols in neuroscience/editorial board, Jacqueline N. Crawley . . . [et al.] Appendix 1, Appendix 1I (2001).

What is claimed is:

1. A method for cryopreservation of human biliary tree stem/progenitor cells comprising
(i) collecting human biliary tree stem/progenitor cells;
(ii) adding a cryopreservation solution to the cells, in which the cryopreservation solution comprises (a) a basal medium comprising lipids, (b) hyaluronans (HA) at a concentration of between about 0.05% and 0.15%, (c) a cryoprotectant, (d) an antioxidant, and (e) albumin at a concentration of between about 1 to 5%; and
(iii) cooling the cells an initial temperature to a final temperature at which the cells are frozen.

2. The method of claim 1, in which the hyaluronan is at a concentration of about 0.1%.

3. The method of claim 1, in which the cryoprotectant is selected from sugar, glycerol, and dimethyl sulfoxide (DMSO), optionally at a concentration of between about 1% and 20%.

4. The method of claim 4, in which the cryoprotectant is DMSO at a concentration of about 10%.

5. The method claim 1, in which the antioxidant is selected from selenium, Vitamin E, Vitamin C, and reduced glutathione.

6. The method of claim 1, in which the albumin is at a concentration of about 3%.

7. The method of claim 1, in which the albumin is purified albumin.

8. The method of claim 1, in which the albumin is human albumin, optionally human plasma-derived albumin or recombinant human albumin.

9. The method of claim 1, in which the cryopreservation solution comprises Kubota's medium, RPMI-1640, DME/F12, or GIBCO's Knockout Serum Replacement Medium.

10. The method of claim 1, in which step (iii) comprises lowering the initial temperature at a rate of about 1° C. per minute until a final temperature is reached.

11. The method of claim 1, in which step (iii) comprises:
(a) cooling cells from the initial temperature to the final temperature of about −80° C. using solid carbon dioxide, or
(b) cooling cells from the initial temperature to the final temperature of about −196° C. using liquid nitrogen.

12. A method of thawing cryopreserved human biliary tree stem/progenitor cells comprising:
(i) thawing cells cryopreserved according to the method of claim 1;
(ii) adding a first buffer solution comprising serum or serum replacement medium;
(iii) separating the cells from the cryopreservation medium and the first buffer solution; and
(iv) resuspending the cells in a second buffer solution comprising serum or serum replacement medium.

13. The method of claim 12, in which the serum is fetal bovine serum or the serum replacement medium is GIBCO's Knockout Serum Replacement Medium or Kubota's medium supplemented with albumin, optionally human serum-derived albumin.

14. The method of claim 12, in which the serum is at a concentration of between about 2% to 20%, optionally between about 10% and 20%, about 10%, or about 20%.

15. The method of claim 12, in which the serum replacement medium comprises albumin at a concentration of between about 1% to 5%, optionally human serum derived albumin.

16. The method of claim 12, in which the culture medium and/or buffer solution comprise a thawing buffer.

17. The method of claim 12, in which step (ii) comprises:
(a) centrifuging the cells;
(b) filtration of the cells through a sieve or filter; or
(c) using French-press type filtration.

18. A method of culturing thawed, cryopreserved human biliary tree stem/progenitor cells comprising:
(i) plating cells thawed according to claim 12;
(ii) culturing the cells in an incubator;
(iii) removing the buffer solution; and
(iv) replacing the buffer solution with a culture medium designed for the growth and/or differentiation of human biliary tree stem/progenitor cells.

19. The method of claim 18, in which step (ii) is conducted for between about 6 to 7 hours.

20. The method of claim 18, in which the culture medium designed for the growth and/or differentiation of human biliary tree stem/progenitor cells comprises Kubota's medium and/or a hormonally defined medium (HDM) for the differentiation of cells (e.g. for lineage restriction to hepatocytes, then. HDM-H).

21. A composition comprising a plurality of cryopreserved human biliary tree stem/progenitor cells produced by the method of claim 1.

22. The composition of claim 21, in which the plurality of cryopreserved human biliary tree stem/progenitor cells are thawed.

23. The composition of claim 22, in which the plurality of cryopreserved human biliary tree stem/progenitor cells are thawed according to the method of claim 12.

24. The composition of claim 21, in which the plurality of cryopreserved human biliary tree stem/progenitor cells are frozen.
